(19) United States
(12) Patent Application Publication
(10) Pub. No.: US 2020/0405773 A1
(43) Pub. Date: Dec. 31, 2020

(54) PROMOTING AND PROTECTING FUNCTIONAL BETA CELL MASS BY SYNTAXIN 4 ENRICHMENT

(71) Applicant: CITY OF HOPE, Duarte, CA (US)

(72) Inventor: Debbie C. THURMOND, Glendora, CA (US)

(73) Assignee: CITY OF HOPE, Duarte, CA (US)

(21) Appl. No.: 16/968,504

(22) PCT Filed: Feb. 8, 2019
(86) PCT No.: PCT/US2019/017360

§ 371 Date: Aug. 7, 2020

Related U.S. Application Data
(60) Provisional application No. 62/628,828, filed on Feb. 9, 2018.

(51) Int. Cl. A61K 35/39; A61K 38/17; C12N 5/071

(52) U.S. Cl. CPC A61K 35/39; C12N 5/0676; A61K 38/1709

(57) ABSTRACT

Disclosed are Syntaxin 4-overexpressing pancreatic islet β-cells, methods of making these cells, and methods of using these cells for promoting and protecting functional β-cell mass, thereby to treat and/or prevent various insulin-related diseases and conditions including diabetes and pre-diabetes. Also disclosed are methods of treating or preventing insulin-related diseases by overexpressing Syntaxin-4.

### PRIORITY CLAIM

This application claims priority to U.S. Provisional Application No. 62/628,828, filed Feb. 9, 2018, which is incorporated by reference herein in its entirety, including drawings.

### GOVERNMENT INTEREST

This invention was made partially with government support under Grant Nos. DK067912, and DK102233, awarded by the National Institutes of Health (NIH). The government has certain rights in the invention.

### BACKGROUND

The regulation of glucose metabolism is a complex physiological process involving the interaction of several hormones, each of which themselves act in many target hormones. Among the hormones involved in glucose metabolism and homeostasis, insulin has the broadest array of functions. Once released by pancreatic p-cells, insulin favors glucose uptake in white adipose tissue and muscle, and suppresses gluconeogenesis in liver. The end result is to decrease blood glucose levels.

Several diseases and disorders or conditions are associated with aberrant insulin secretion or signaling, including type 1 diabetes (T1D), type 2 diabetes (T2D), chronic pancreatitis, pancreatectomy, insulin resistance, prediabetes, and age-related insulin resistance. These diseases or conditions may lead to serious life-threatening complications; however, treatment or prevention of these diseases or conditions is not satisfactory.

For example, over 100 million people in the US suffer from type 2 diabetes (T2D) or are pre-diabetic (35, 36). Additionally, half of pre-diabetic individuals are at risk of developing T2D within 5 years (35, 37). Prediabetic individuals suffer from elevated blood glucose levels ranging from 100-125 mg/dl or impaired glucose tolerance (38). Importantly, pre-diabetes is reversible; however, current treatments are limited to exercise, dietary modifications (39), and anti-hyperglycaemic drugs that can increase insulin sensitivity, like metformin (40). Biguanides like metformin have undesirable side effects, and as T2D becomes more prevalent, there is yet to be a suitable therapeutic on the market that is able to not only mediate diabetic symptoms, but also reverse insulin resistance to prevent the onset of frank T2D.

Thus, there is a need to develop methods for treating or preventing diseases and disorders or condition are associated with aberrant insulin secretion or signaling.

### BRIEF DESCRIPTION OF THE DRAWINGS

FIGS. 1A-1C depict the correlation between Syntaxin 4 (hereinafter Stx4 or Syn4) and susceptibility to dysregulated glucose homeostasis. FIG. 1A: Stx4 heterozygous knockout mice (Syn4-Het) subjected to a low dose streptozotocin (STZ) treatment (35 mg/kg body weight for 5 consecutive days, IP) showed higher glucose intolerance (STZ-Syn4 Het) than wild-type control mice (WT) exposed to the same STZ treatment (STZ-WT) 5 days later (10 days after 1st injection). N=4 mice per genotype, 1 mg/ml glucose injection; *p<0.05 vs STZ-treated WT mice). AUC, Area under the curve. FIG. 1B: Islets from pre-type 1 diabetic NOD mice (NOD) showed Stx4 protein deficiency compared to age matched diabetes-resistant NOR mice (NOR). Stx4 content was assessed by immunoblot analysis, tubulin used as loading control; *p<0.002. AU, arbitrary unit. FIG. 1C: Human islet acute insulin release decreased as a function of increasing age of the islet donor. Peak amplitude of first-phase GSIS from human islet perifusion data (AIR) was stratified by age of the human islet donors, using data from 8 donors; inverse association of peak amplitude of 1st phase GSIS and human islet donor age, R²=0.84, p=0.0014.

FIGS. 2A-2C show that Stx4 enrichment selectively in the mouse islet β-cell improves glucose tolerance and islet function in response to glucose. FIG. 2A: Design strategy of the βTG-Stx4 mouse model. Rat insulin promoter (RIP) or mouse insulin promoter (MIP)-rtTA^{+/-} heterozygous transgenic mice were crossed to TRE-Stx4^{+/-} heterozygous transgenic mice to generate tetracycline/doxycycline (dox)-inducible β -cell specific Stx4 overexpression in the islets in adult mice (βTG-Stx4). Presence of doxycycline triggered rtTA-mediated expression of the transgene. FIG. 2B: RIP-based βTG-Stx4 mice show dox-inducible Stx4 overexpression in islets and without expression in hypothalamus (n=3 mice per group, *p<0.05 Ctrl (−dox-βTG-Stx4) vs βTG-Stx4 (+dox-βTG-Stx4)). FIG. 2C: β-cell specific Stx4 overexpression was confirmed in isolated mouse islet β-cells by immunofluorescent confocal microscopy colocalization of insulin; no colocalization was observed in glucagon-stained α-cells (n=3-5 islets per 3 βTG-Stx4 mice each).

FIG. 3 shows protein expression levels of Stx4 and its binding partners in various tissues from βTG-STX4 mice compared with control littermates. Heart, lung, liver, kidney, spleen, skeletal muscle (SKM), epigonadal fat and pancreatic islet tissues were isolated from female βTG-Stx4 mice treated without (Ctrl) or with dox (Stx4) and homogenized. Twenty μg of protein were resolved by SDS-PAGE and immunoblotted with antibodies for Stx4, Munc18c and SNAP23. The intensity of Stx4 expression was normalized to Ponceau S staining. Immunoblots are representative at least three independent sets of tissues per group. Bars represent the mean ±SE in 3 independent sets of tissues.

FIG. 4A shows intraperitoneal glucose tolerance tests (IPGTT) of female Ctrl (yellow) vs. dox-induced βTG-Stx4 mice (green); area under the curve (AUC) analyses of IPGTT testing, *p<0.05, n=6−11. FIG. 4B shows Stx4 expression and glucose tolerance testing in single transgenic mice. Area under the curve analyses from IPGTT tests of single transgenic mice treated without or with doxycycline: (−)Dox; TRE-Stx4/+, (+)Dox; TRE-Stx4/+, (−)Dox; RIP-rtTA/+, (+)Dox; RIP-rtTA/+; n=3 female mice per group.

FIGS. 5A-5C show Stx4 expression and glucose tolerance testing in the second TRE-Stx4 founder line. FIG. 5A: Stx4 expression in a second founder line of TRE-Stx4 mice (founder #9407) in islets and hypothalamus; data represent 3 female mice of each genotype/dox treatment shown. FIG. 5B: Stx4 protein abundances normalized to tubulin in islets, n=2 double transgenic female mice per Ctrl (−dox) or βTG-Stx4 (+dox) group. FIG. 5C: Dox-induced βTG-Stx4 mice IPGTT testing of Ctrl (−dox) or βTG-Stx4 (+dox) female mice of the second founder line (#9407); *p<0.05, n=3−4.

FIG. 6 shows intraperitoneal insulin tolerance tests (IPITT) of female Ctrl (yellow) vs. dox-induced βTG-Stx4 mice (green); n=6-11.

FIGS. 7A-7C show Stx4 overexpression driven by the MIP (mouse insulin promoter)-rtTA. FIG. 7A: Stx4 expression in islets and hypothalamus from TRE-Stx4 (first founder line)×MIP-rtTA mice; data represent 3 female mice of each genotype/dox treatment shown. FIG. 7B: Stx4 protein abundances normalized to tubulin in islets, n=2 double transgenic female mice per Ctrl (−dox) or MIP-based βTG-Stx4 (+dox) group. FIG. 7C: IPGTT testing of MIP-based Ctrl (−dox) or βG-Stx4 (+dox) male mice; *p<0.05, n=3−4, performed before STZ treatment.

FIG. 8A shows serum insulin content under fasted or glucose-stimulated (Gluc, 10 min) in female Ctrl or dox-induced βTG-Stx4 mice; n=6−8 per group, *p<0.05 glucose-stimulated Ctrl vs βTG-Stx4. FIG. 8B shows ex vivo static glucose stimulated insulin secretion (GSIS) from islets isolated from Ctrl and dox-induced βTG-Stx4 female mice. Basal secretion (Bsl) remains normal; (n=4-5 sets of islets per group, *p<0.05 Ctrl vs βTg-Stx4 under glucose-stimulated conditions).

FIGS. 9A-9F show that Stx4 abundance positively correlates with improved glucose tolerance and functional β-cell mass. FIG. 9A: Partial Stx4 deficiency heightens susceptibility to MLD-STZ-induced dysregulation of glucose homeostasis. Stx4 heterozygous knockout male mice (Stx4 (+/−)) and wild-type (Wt) littermate mice were subjected to a multiple low dose streptozotocin protocol (MLD-STZ, 35 mg/kg body weight for 5 consecutive days). Ten days post MLD-STZ protocol initiation, mice were subjected to IPGTT (1 g glucose/kg body weight), n=5-7 male mice per genotype; *p<0.05 vs MLD-STZ-treated Wt mice. AUC, area under the curve. FIG. 9B: TUNEL immunofluorescent staining and quantification of TUNEL-positive β-cells (% of total β-cells) from pancreata of MLD-STZ-treated Wt or Stx4 (+/−) mice (n=3 per genotype, p=0.12). FIG. 9C: Dox-induced male βTG-Stx4 mice are protected from MLD-STZ-induced glucose intolerance 24 days post initiation of the MLD-STZ protocol. IPGTT was performed using 2 g of glucose/kg body weight (n=7-8 dox-induced (βTG-Stx4) or non-induced (Ctrl)); *p<0.05 vs Ctrl mice. FIG. 9D: Fasting (6 h) blood glucose levels measured from MLD-STZ treated Ctrl or βTG-Stx4 male mice. FIG. 9E: TUNEL immunofluorescent staining and quantification of TUNEL-positive β-cells (% of total β-cells) from pancreata of MLD-STZ-treated Ctrl or βTG-Stx4 mice (n=5-6 pancreata per group); *p<0.05 vs MLD-STZ-treated Ctrl mice. Bar=20 μm. Arrowheads denote representative images of TUNEL+ cells. FIG. 9F: Islet β-cell mass is preserved in MLD-STZ-treated dox-induced βTG-Stx4 male mice; n=3-4 male mice pancreata per group; *p<0.05 Ctrl vs MLD-STZ treated Ctrl.

FIGS. 10A-10C show that Stx4 overexpression driven by the MIP (mouse insulin promoter)-rtTA. FIG. 10A: Re-testing of the same male mice 24 days after STZ treatment (35 mg/kg body weight for 5 consecutive days). FIG. 10B: Fasting blood glucose was measured from STZ-treated male Ctrl or βTG-Stx4 mice (n=3-4 mice per group). FIG. 10C: Quantification of TUNEL-positive β-cells (% of total β-cells) from fixed pancreases of STZ-treated Ctrl or βTG-Stx4 mice; *p<0.05 vs. STZ-treated Ctrl mice.

FIGS. 11A-11E show that selective over-expression of Stx4 in human islet β-cells improves function and exerts protection from proinflammatory cytokine-provoked apoptosis. FIG. 11A: Human islets transduced to express control (Ad-RIP-Ctrl) or Stx4 (Ad-RIP-Stx4) to selectively enrich Stx4 in β-cells of the islets show no overexpression in glucagon-stained α-cells. Arrows denote location of α-cells, superimposed on Stx4-stained images captured by dual-laser scanning confocal Immunofluorescent microscopy. FIG. 11B: Ad-RIP-Stx4-overexpression in human islets promotes and enhanced stimulation index response (stimulation index=glucose-stimulated/basal insulin secretion) in both phases in perifusion analyses (n=5 donor islet sets per group, *p<0.05 Ad-RIP-Ctrl vs. Ad-RIP-Stx4). FIG. 11C: Human islets were transduced with Ad-RIP-Ctrl or Ad-RIP-Stx4 and subsequently exposed to proinflammatory cytokine cocktail (TNFα, IL-1β, IFN [symbol] ) for 16 h and apoptosis assessed by immunoblotting detergent cell lysate proteins for cleaved caspase 3 (CC-3). Abundance of CC-3 in cytokine (+Cyt)-exposed islets was normalized to tubulin, and fold over control (−Cyt) determined in 3 sets of donor islets per treatment group; *p<0.05 Ad-RIP-Ctrl Cyt vs. Ad-RIP-Stx4 Cyt. FIG. 11D: Human EndoC-βH1 β-cells were transduced and assessed as described in panel C above. Data represent the average of three independent passages of cells per group, *p<0.05 Ad-RIP-Ctrl Cyt vs Ad-RIP-Stx4 Cyt. FIG. 11E: MIN6 β-cells were transduced, exposed to proinflammatory cytokines for 24 h and assessed as described in panel FIG. 11C above. Data represent the average of three independent passages of cells per group, *p<0.05 Ad-RIP-Ctrl Cyt vs Ad-RIP-Stx4 Cyt.

FIGS. 12A-12D show RNA sequencing analysis of β-cell specific Stx4 over-expression in human islets. FIG. 12A: Three independent batches of human donor islets were transduced with either Ad-RIP-Stx4 or Ad-RIP-Ctrl and RNA extracted for RNA sequencing analyses (RNAseq). Heat map analysis shows clustering for differentially expressed genes grouped by virus type. FIG. 12B: Quantitative Real time-PCR was performed using the same RNA used for RNAseq to confirm Stx4 overexpression and two major hits, CXCL9 and CXCL10. FIG. 12C: RNAseq data were used to generate an Ingenuity Pathway Analysis network. FIG. 12D: Human islets transduced as described in FIG. 12A above were harvested for subcellular fractionation into nuclear and cytoplasmic fractions, and localization of total (t)-NF-κB and phosphorylated (p)-NF-κB proteins therein assessed by immunoblotting. Tubulin and TBP served as loading controls. Stx4 was found only in the non-nuclear fraction; Stx4 expression validated the virus transduction. Data represent the averages from three independent batches of human donor islets; *p<0.05 Ad-RIP-Ctrl vs Ad-RIP-Stx4 nuclear fraction of p-NF-κB). STX4, Syntaxin 4; SNAP25, Synaptosomal-associated protein 25; NGF, Nerve growth factor; STMN4, stathmin 4; ERK, extracellular-signal-regulated kinase; INS, Insulin; MAPK8, Mitogen-Activated Protein Kinase 8; IFNG, Interferon gamma, IL1B; Interleukin 1 Beta; NF-kB, Nuclear factor-κB; IKBKB, Inhibitor of nuclear factor kappa B kinase subunit beta; TNF, tumor necrosis factor; CXCL, Chemokine (CXC motif) ligand; TAC1; Tachykinin, Precursor 1; TACR1, Tachykinin Receptor 1.

FIG. 13 shows that Stx4 does not co-immunoprecipitate with NF-kB in human EndoC-βH1 cells. Human EndoC-βH1 cells were transduced with Ad-RIP-Ctrl or Ad-RIP-Stx4 and 48 h later were harvested in detergent lysis buffer for use in anti-Stx4 or anti-NF-kB co-immunoprecipitation reactions (lanes 3-6). Stx4 overexpression was confirmed in lysates (lanes 1-2); NF-kB protein levels did not change with Stx4 overexpression. Unbound (flow-through, FT) proteins confirmed the antibody specific depletion of Stx4 (lanes 7-8) or NF-kB (lanes 9-10). Stx4 IP failed to co-immunoprecipitate NF-kB (lanes 3-4), and NF-kB IP failed to co-immunoprecipitate Stx4 (lanes 5-6). Results are representative of 6 independent set of immunoprecipitation experiments.

FIG. 14 shows an example of a vector construct for overexpression of Stx4. The vector has a sequence encoding a human insulin promoter and human Stx4 cDNA.

FIGS. 15A-15I show that selective Syntaxin 4 (STX4) enrichment in skeletal muscle boosted peripheral insulin sensitivity and indirectly improved pancreatic functionality. Phenotyping of female STX4 transgenic mice (Skm STX4) treated with or without 2 mg/ml doxycycline (control mice were double transgenic without Dox-Black, and Skm STX4 were double transgenic mice treated with Dox-Green). Testing was performed on 4-6 months old female C57/B6 mice. FIG. 15A shows a gene map of the Tet-On inducible model of STX4 overexpression. FIG. 15B shows that immunoblot of mouse skeletal muscle demonstrated an increase in STX4 expression with Dox treatment. Heart lysates were used as negative control. FIG. 15C shows insulin tolerance test (ITT). Bar graph shows data expressed as area over the curve (AOC). FIG. 15D shows glucose tolerance test (IPGTT). Bar graph of data expressed as area under the curve (AUC). FIG. 15E shows body weight of the mice. FIG. 15F shows body composition of the mice. Lean mass in gray (tall bars) and fat mass in yellow (low bars), expressed as percentage of total body weight. FIG. 15G shows 6-hour fasted serum insulin levels. FIG. 15H shows GSIS of ex-vivo pancreatic islets, normalized to insulin content (n=3 mice/group). FIG. 15I shows β-cell mass of CTRL and STX4 mice. Data are mean ±SE of 5-7 mice. *p<0.05 (Students t-test, or One-Way ANOVA).

FIGS. 16A-16H show phenotypic data from chow-fed male control and Skm-STX4 mice. FIG. 16A shows male mouse ITT represented as percentage of basal blood glucose. FIG. 16B shows male mouse GTT. FIG. 16C shows insulin content for ex vivo islet GSIS. 3 mice per group. FIGS. 16D-16H show plasma metabolites of overnight fasted mice. All data are representative of n=5-9 per group.

FIGS. 17A-17I show that STX4 enrichment in skeletal muscle of obese HF-fed mice reversed insulin resistance. FIG. 17A shows schematic flow: mice were placed on a 45% high fat diet (HFD) for 10 weeks until they became insulin resistant (defined as: mice that don't reach below 60% of basal blood glucose by 60 mins), as measured by ITT (18 weeks). The STX4 transgene was then induced using doxycycline in the food in half the mice, while the other half remained on only HFD. After 5 weeks the mice were tested for insulin response by ITT again (22 weeks). FIG. 17B shows ITT pre- (orange dots- HFD only) and post-induction (green dots, HFD with STX4 induction) of HFD mice at 18 weeks and then again at 22 weeks. HFD mice before and after STX4 induction. Data represented as total area over the curve (AOC), each mouse can be traced to itself. AOC was calculated for all HFD (n=8) and HFD+Dox (n=7) mice at 18 weeks and then again at 22 weeks. FIG. 17C shows control mice on HFD at 18 weeks and then again at 22 weeks. FIG. 17D shows ITT data of mice at 18 weeks (orange) and then again the same mice at 22 weeks (green) compared to chow fed controls (Black). FIG. 17E shows percentage of body weight change over time in HFD and HFD+STX animals. FIG. 17F shows body weight of mice at 22 weeks. FIG. 17G shows body composition of mice at 22 weeks. FIG. 17H shows average daily calories consumption per week over the paradigm period. FIG. 17I shows serum insulin levels of mice at 24 weeks, after a 6 hour fast. Statistics calculated using one-way ANOVA or student t-test *p<0.05, Chow n=3, HFD, HFD+Dox n=7.

FIGS. 18A-18I show HFD GTT and plasma metabolites of chow-fed, HFD and HFD+STX4 male mice. FIG. 18A shows 6-hour fast GTT on chow (black), HFD (orange) and HFD+ STX4 (green) male mice at 23 weeks old. FIGS. 18B-18I show the levels of various plasma metabolites. Fasted overnight. Statistics are one-way ANOVA, *p>0.05.

FIGS. 19A-19D show that STX4 enrichment in HF-fed mice normalized energy source utilization and spontaneous physical activity. Metabolic caging analysis of 8-week old chow fed mice (black), 17-week old HF-fed mice (orange) and 22-week old post treatment HF-fed mice with STX4 enrichment (green) over a 48-hour period. FIG. 19A shows respiratory exchange ratio (RER). FIG. 19B shows average hourly spontaneous activity measured as distance K (cm/hr) during the day (6am-6pm) and night (6pm-6am). FIG. 19C shows average energy expenditure per hour normalized to body weight. All data representative of 7-16 mice per group, statistics: p<0.05 One-way or two-way ANOVA. FIG. 19D shows extracellular flux analysis (Seahorse by Agilent) involving the inhibition of mitochondrial complexes to assess mitochondrial function and oxidative phosphorylation of HFD (orange) and HFD+STX4 (green) mice to assess mitochondrial function. Data from n=3 experiments and mice normalized to basal. Right panel: Bar graph of the change in maximal respiration between the HFD and STX4 groups.

FIGS. 20A-20F show that STX4 improved mitochondrial function via mitochondrial morphology and dynamics changes. FIG. 20A shows mitochondrial DNA (mtDNA) of chow fed mice (black) and those expressing STX4 (green) measured by comparing relative levels of Cox1 to 18 s ribosomal DNA. Mitochondrial DNA (mtDNA) in (left panel) chow female controls (black) and STX4 induced (green), and (right panel) male chow (black) hf-fed (orange) and hf-fed +STX4 males (green). n=6-8/group. DNA extracted from hindlimb. FIG. 20B shows whole hindlimb whole cell lysate immunoblot of electron transport chain complex, comprised of Complex I, II, III, IV and V, in chow fed CTRL and STX4 mice, with kit positive control. Ponceau used as loading control. Representative of n=6 mice. FIG. 20C shows citrate synthase activity after 4.5 min reaction in hindlimb muscle (top panel chow females, bottom panel HFD males). FIG. 20D shows representative TEM images of tibialis anterior intramyofibullar (IMF) mitochondria at magnification of 4400X (top) and 11000X (bottom), in CTRL, STX4 overexpressing, HFD and HFD+STX4 mice. FIG. 20E shows immunogold staining of IMF mitochondria in STX4 overexpression chow fed mouse, stained with STX4 antibody. STX4 clusters highlighted in yellow arrows, example of mitochondria outlined in red. FIG. 20F shows immunoblot of Drp1 protein in chow ctrl or chow STX4 hindlimb whole extract. Bar graph is quantitation of normalized immunoblots, n=6/ group. All images are representative of 3-6 animals/group with 4-6 fields of view taken per mouse muscle section.

FIGS. 21A-21B show control mitochondrial analyses and EM imaging. FIG. 21A shows citrate synthase assay in tibialis anterior muscle of chow fed (left panel) and high fat fed mice (right panel). FIG. 21B shows EM immunogold labeling validation using blocking peptide.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The term “subject” as used herein refers an individual having impaired glucose homeostasis resulting from an insulin-related disease or condition including but not limited to type 1 diabetes (T1D), type 2 diabetes (T2D), chronic pancreatitis, pancreatectomy, insulin resistance, prediabetes, and age-related insulin resistance. A subject can be any individual mammal suffering from an insulin-related disease or condition, including but not limited to human, canine, rodent, primate, swine, equine, sheep, and feline. “Insulin-related disease or condition” refers to any disease or condition characterized by an insulin deficiency or insulin resistance. In a particular embodiment, the subject is human.

As used herein, the term “allogenic,” refers to tissue or cells from a distinct subject of the same species. The term “xenogenic” refers to tissue or cells from a subject of another species. The terms “autogenic” and “autologous” refer to tissue or cells derived from a subject, and re-introduced into the same subject.

As used herein, an “effective amount” refers to an amount of a substance or composition sufficient to bring about improved whole body glucose. An effective amount, or effective dose, can be administered in one or more administrations. The precise determination of an effective amount will be affected by factors individual to each subject, including, but not limited to, the subject's age, size, type and extent of insulin-related disease or condition, method of administration, whether the substance or composition is administered alongside conventional therapy or on its own, and the desired result. It will be known to one of skill in the art how to determine an effective amount for a particular subject.

As used herein, a “promoter” means a nucleotide region comprising a nucleic acid (i.e., DNA) regulatory sequence, wherein the regulatory sequence is derived from a gene that is capable of binding RNA polymerase and initiating transcription of a downstream (3′-direction) coding sequence. Transcription promoters can include “inducible promoters” (where expression of a nucleic acid sequence operably linked to the promoter is induced by an analyte, cofactor, regulatory protein, etc.), “repressible promoters” (where expression of a polynucleotide sequence operably linked to the promoter is repressed by an analyte, cofactor, regulatory protein, etc.) and “constitutive promoters” (where expression of a polynucleotide sequence operably linked to the promoter is unregulated and therefore continuous).

As used herein, “operably linked” means that elements of an expression sequence are configured so as to perform their usual function. Thus, control sequences (i.e., promoters) operably linked to a coding sequence are capable of effecting expression of the coding sequence. The control sequences need not be contiguous with the coding sequence, so long as they function to direct the expression thereof. Thus, for example, intervening untranslated, yet transcribed, sequences can be present between a promoter and a coding sequence, and the promoter sequence and still be considered “operably linked” to the coding sequence.

As used herein, the terms “providing,” “administering,” “introducing,” “delivering,” “placement,” and “transplanting” may be used interchangeably herein to refer to the placement of a cell or nucleic acid described herein in a subject.

As used herein, “pharmaceutically-acceptable excipient” means a pharmaceutically acceptable material, composition or vehicle involved in giving form or consistency to a pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must be of sufficiently high purity to render it pharmaceutically-acceptable.

A “pharmaceutically acceptable carrier” as used herein refers to a pharmaceutically acceptable material, composition, or vehicle that is involved in carrying or transporting an agent or cell of interest from one tissue, organ, or portion of the body to another tissue, organ, or portion of the body. Such a carrier may comprise, for example, a liquid, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, or some combination thereof. Each component of the carrier is “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) natural polymers such as gelatin, collagen, fibrin, fibrinogen, laminin, decorin, hyaluronan, alginate and chitosan; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as trimethylene carbonate, ethyl oleate and ethyl laurate; (13) agar; (14) buffering agents, such as magnesium hydroxide and aluminum hydroxide; (15) alginic acid (or alginate); (16) pyrogen-free water; (17) isotonic saline; (18) Ringer's solution; (19) alcohol, such as ethyl alcohol and propane alcohol; (20) phosphate buffer solutions; (21) thermoplastics, such as polylactic acid, polyglycolic acid, (22) polyesters, such as polycaprolactone; (23) self-assembling peptides; and (24) other non-toxic compatible substances employed in pharmaceutical formulations such as acetone.

The terms “treat,” “treating,” and “treatment” as used herein with regard to a cancer condition refer to alleviating the condition partially or entirely, or eliminating, reducing, or slowing the development of one or more symptoms associated with the condition.

Both type 1 and type 2 diabetes are linked to inflammation related to the damaging effects of pro-inflammatory cytokines upon pancreatic islet β-cells (1, 2). Evidence suggests that dysfunctional insulin release from the β-cell precedes the onset of type 1 diabetes mellitus (T1D) (3). One of the rate-limiting features of β-cell insulin release is the abundance of soluble N-ethylmaleimide sensitive factor attachment protein receptors (SNAREs), with accumulating evidences pointing to a paucity of SNARE proteins as an underlying cause of β-cell dysfunction in type 2 diabetes (T2D) (4-7).

Two types of target membrane (t)-SNARE proteins, syntaxins and SNAPs, assemble with one vesicle (v)-SNARE protein to form a heterotrimeric SNARE core complex, docking and fusing the insulin granule at the plasma membrane to facilitate insulin release from the β-cell. Specific isoforms Syntaxin 1A (Stx1A) and Syntaxin 4 (Stx4) oversee glucose-stimulated insulin secretion (GSIS), pairing with v-SNAREs VAMP8 (8) and VAMP2 (9), respectively, along with SNAP25 (10). Of these SNAREs, Stx4 was identified as a T1D candidate protein, predicted by in silico phenome-interactome network analysis (11), and the Stx4 gene was localized within T1D susceptibility region Iddm10 (T1Dbase.org). In dysfunctional islets from human T2D individuals, Stx4 plus other SNARE proteins are found to be 50-75% reduced (5, 9, 12), yet the replenishment of Stx4 alone is capable of restoring normal GSIS to dysfunctional T2D human islets (9). In a minimal islet transplant model of diabetes in vivo, Stx4-enriched mouse islets were better capable of attenuating diabetes over the entire experimental duration. At present, it remains unknown whether Stx4 function in β-cell GSIS is the primary or sole mechanism to underpin the restored GSIS phenomenon and/or the minimal islet transplant phenomenon, due to the limitations of the prior studies; Stx4 overexpression systems used in human islets and mice were not β-cell specific.

Current treatment of hyperglycemia in T1D patients involves exogenous insulin delivery, which results in constitutive and non-regulated insulin delivery. Consequently, hypoglycemic episodes are a significant health hazard in T1D. Islet transplantation can more effectively mimic endogenous insulin secretion, and has become an exciting alternative to the exogenous insulin injection. However, a major constraint for islet transplantation to patients is that each transplant requires islets from several donors.

STX4 can be used for treating and/or preventing diabetes or pre-diabetes. As demonstrated herein, STX4 overexpression in skeletal muscle of high fat fed mice was able to reverse insulin resistance. Additionally, chow fed mice overexpressing STX4 selectively in skeletal muscle were found to have enhanced glucose tolerance, insulin sensitivity, and reduced fasted serum insulin compared to control mice. These results suggest that STX4 functions in the skeletal muscle to regulate glucose homeostasis, and restore peripheral insulin sensitivity, and that STX4 is a potential therapeutic to remediate pre-diabetes.

As described herein, β-cell selective expression systems were utilized to demonstrate that Stx4 plays an unexpected role in controlling the gene expression profiles. Stx4 serves as an anti-apoptotic, preserving β-cells from inflammatory-induced destruction via tempering the NF-kB-induced CXCL9 and CXCL10 inflammatory program. Human islet β-cells enriched for Stx4 were protected from pro-inflammatory cytokine-induced apoptosis, coordinate with a retention of NF-κB in the cytosolic compartment and reduced expression of chemokine ligands affiliated with diabetes, namely CXCL9 and CXCL10. Strikingly, the STX-diabetes-resistant phenotype of the βTG-Stx4 mice suggests potential for Stx4 enrichment as diabetes treatment and prevention.

Induction of STX4 selectively in muscle of high fat fed, insulin resistant mice restores insulin sensitivity to the level of chow fed mice. These mice also show a restoration of normal physical activity. Mechanistically, the induction of STX4 in skeletal muscle of mice on HFD also imparts improved mitochondrial function in the muscle, by mediating mitochondrial dynamics and morphology. Skeletal muscle-specific overexpression of STX4 phenocopies the global STX4 overexpressing mice, pointing to the role of STX4 in skeletal muscle as being the primary driver in regulating insulin sensitivity. STX4 enrichment in the skeletal muscle after high fat diet feeding remediates insulin resistance, suggesting that STX4 can reverse insulin resistance and serve as a T2D therapeutic.

In pre-diabetics and healthy individuals, peripheral tissues like skeletal muscle are essential for glucose clearance. Over 80% of glucose clearance occurs via the skeletal muscle (41); however, no directed therapeutics to restore skeletal muscle function in pre-diabetics exist. Disclosed herein is a potential new therapeutic for pre-diabetes; Syntaxin 4 (STX4), which is capable of reversing skeletal muscle insulin resistance in a skeletal muscle specific STX4 inducible overexpression mouse model. STX4 is a key modulator of insulin stimulated glucose uptake (42), and it does so via interaction with other SNARE proteins to facilitate the fusion of glucose transporter 4 (GLUT4) bound vesicles with the plasma membrane, placing GLUT4 at the cell surface to enable glucose uptake into the muscle cell (43). Although part of a multi-protein SNARE complex, STX4 has been found as a rate limiting factor in GLUT4 vesicle fusion to the plasma membrane (44), and a regulator of this process in skeletal muscle (45). Mouse models with global overexpression of STX4 had to a 2-fold increase in glucose uptake and increase in GLUT4 at the plasma membrane (46). It has been shown that STX4 is decreased in skeletal muscle of insulin resistant and type 2 diabetic individuals (47), suggesting that enrichment of STX4 to replenish levels can be used as a directed therapeutic to restore skeletal muscle function in pre-diabetics. Although STX4 enrichment is beneficial in insulin resistant mouse models, the tissue specific effects of STX4 enrichment have yet to be delineated largely due to the lack of tissue specific models of STX4. Disclosed herein is a new transgenic mouse model that selectively and inducibly expresses STX4 in the skeletal muscle. This model was used to assess and demonstrate the ability of STX4 to reverse high-fat diet induced peripheral insulin resistance in C57B/6 mice. The targeted overexpression of STX4 in skeletal muscle tissue was sufficient to remediate insulin sensitivity and glucose tolerance, under high fat conditions.

As demonstrated in the working examples provided herein, certain β-cells with Stx4 overexpression showed longer life expectancy and better protection from inflammatory damage than β-cells without Stx4 overexpression. Thus, fewer islets would be required for each transplantation, and more patients can benefit from the islet transplantation treatment. β-cells with Stx4 overexpression, and with increased life expectancy and/or lower susceptible to inflammation damage may further be useful for improving whole body glucose homeostasis in a subject, and/or preventing, delaying, and/or treating insulin-related diseases and conditions in the subject (e.g., type I and II diabetes, chronic pancreatitis, pancreatectomy, insulin resistance, prediabetes, and age-related insulin resistance), and/or improving the lifespan of a subject.

This disclosure relates to remediation of insulin resistance and reversal of pre-diabetes by the enrichment of a protein in skeletal muscle selectively. As disclosed herein, STX4, a SNARE protein involved in GLUT4 translocation and glucose uptake in the skeletal muscle, is capable, when excess, to remediate insulin resistance induced by a high fat diet. The insulin tolerance test showed improved insulin sensitivity after STX4 induction while still maintained on a high fat diet. Thus, STX4 is capable, even in the continued intake of diabetogenic stress to act as a therapeutic for insulin resistance (pre-diabetes), and STX4 has the potential, even under diabetogenic conditions to ameliorate hyperinsulinemia and increase insulin secretion by the pancreatic islets. The therapeutic effect may be due to STX4 localizing at the mitochondrial membrane, and mediating mitochondrial dynamics, reverting vacuolized, and fragmented mitochondria in the HF-fed mice, back to structured, round and organized organelles.

As demonstrated in the working examples, the most insulin resistant mice were used for doxycycline-induced STX4 enrichment. This approach is the most stringent way to assess the therapeutic potential of STX4. The use of randomized mice of varying insulin sensitivities would have not produced the compelling data because every mouse placed on a high fat diet supplemented with doxycycline became more insulin sensitive in 4-5 weeks. Although all mice were placed on the 45% high fat diet for the same duration, there was a lot of heterogeneity in the HFD paradigm, where only some mice get insulin resistant, and to varying degrees (71). In addition, the use of the 45% diet is more physiologically similar to the diabetogenic western diet (72, 73), whereas the more potent 60% high fat diet is not physiologically relevant, and causes irreversible pancreatic β-cell damage and demise.

Furthermore, the working examples demonstrate the ability of STX4 to induce spontaneous activity in mice on a HFD, showing a significant increase in the distance moved over 2 days. This suggests that STX4 has the ability to act as an exercise mimetic in addition to being an insulin sensitizer. This is further evidenced by the improvement in mitochondrial function, and the changes in mitochondrial structure. Exercise induces mitochondrial elongation (69), and as shown in Chow fed mice overexpressing STX4, they also had elongated and enlarged mitochondria. Furthermore, exercise trained mice not only showed improved mitochondrial function but also increased spontaneous physical activity levels compared to untrained controls (56), as shown in the seahorse experiment. This result was similar to the mice on HFD+STX4 compared to HFD mice. Exercise was shown to be an important lifestyle intervention in mediating pre-diabetes and T2D, as it was shown to improve glucose tolerance and insulin sensitivity, similar to the capability of STX4. However, mice remained obese after STX4 induction, whereas exercise caused weight loss and reduced percentage of abdominal body fat (56). This could be due to the fact that the mice were maintained on the high fat diet, and they displayed no changes in caloric intake, so the spontaneous physical activity did not reduce their caloric load sufficiently enough to lead to weight loss. Should STX4 be induced in conjunction with reverting the mice back to a chow diet, the weight loss effects may be observed with a complete exercise mimetic (but without the actual exercise component).

The working examples demonstrate that STX4 localizes to the mitochondrial outer membrane, when it has previously only been shown to localize to the plasma membrane (74-76). Not only does it localize to the mitochondrial membrane STX4 impacts Drp1 to reduce its abundance and in turn inhibits mitochondrial fission. This would explain the ability of STX4 to remediate high-fat diet induced insulin resistance, as a hallmark of HFD is increased mitochondrial fission and mitophagy (77).

### β-cells that Overexpress Stx4

Provided herein are populations of β-cells that overexpress Stx4 (“the β cell populations”). The β-cell populations can be used in research methods or in treatment and prevention of various insulin-related conditions including diabetes.

Also provided herein are pharmaceutical compositions comprising an effective amount of islet β-cells overexpressing Stx4, wherein the β-cells overexpressing Stx4 have enhanced insulin secretory capacity, improved life expectancy, and/or improved protection against pro-inflammatory cytokine-induced apoptosis compared to normal islet β-cells. In certain embodiments, the β-cells overexpressing Stx4 show upregulation of one or more genes related to decreasing oxidative stress, increasing cell proliferation, and/or increasing cell survival. In certain embodiments, the β-cells overexpressing Stx4 show upregulation of one or more genes such as HSPA6. In certain embodiments, the β-cells overexpressing Stx4 show downregulation of one or more genes related to apoptosis and/or inflammation. In certain embodiments, the β-cells overexpressing Stx4 show downregulation of one or more genes selected from the group consisting of CXCL9, CXCL10, and CXCL11.

### Methods for Improving the Life Expectancy and/or Protection from Inflammatory Damage of β cells

Provided herein are methods for improving the life expectancy of islet β-cells in a subject such as a mammal comprising enriching Stx4 in the islet β-cells.

Provided herein are also methods for protecting islet β-cells in a subject such as a mammal from inflammatory damage comprising enriching Stx4 in the β-cells.

In certain embodiments, the methods disclosed herein further comprise reducing apoptosis of the islet β-cells.

In certain embodiments, the methods disclosed herein further comprise upregulating one or more genes in the islet β-cells that are related to decreasing oxidative stress, increasing cell proliferation, and/or increasing cell survival. In certain embodiments, the one or more genes upregulated include HSPA6.

In certain embodiments, the methods disclosed herein further comprise downregulating one or more genes related to apoptosis and/or inflammation. In certain embodiments, the one or more genes downregulated are selected from the group consisting of CXCL9, CXCL10, and CXCL11.

In certain embodiments, enriching Stx4 in the islet β-cells comprises inducing Stx4 overexpression in β-cells by cell therapy, gene therapy, or a combination thereof, as disclosed herein.

### Cell Therapy

Wherein overexpression of Stx4 in β-cells in a subject is induced by cell therapy, the cell therapy comprises administering to the subject a population of β-cells overexpressing Stx4. In certain embodiments, the β-cells overexpressing Stx4 have longer life expectancy and better protection from inflammatory damage compared to β-cells without Stx4 overexpression. In certain embodiments, overexpression of Stx4 in β-cells is achieved by introducing into the β-cells at least one nucleic acid encoding Stx4. In certain embodiments, the at least one nucleic acid has a sequence identity at least 80% identical to one nucleic acid selected from the group consisting of the coding sequence of the human Stx4 gene or the human Stx4 gene with a L173A/E174A mutation, which makes it constitutively active in an unfolded state, as described in JBC, 2S2: 16553-16566, (2007), which is incorporated herein by reference. Constitutively active Stx4 provides for more robust insulin secretion.

In certain embodiments of the methods disclosed herein, the at least one nucleic acid has a sequence identity selected from the group consisting of: at least 80%; at least 85%; at least 90%; at least 95%; at least 96%; at least 97%; at least 98%; at least 99%; and 100% identical with one nucleic acid selected from the group consisting of the human Stx4 gene and the human Stx4 gene with a L173A/E174A mutation.

One or more nucleic acids can be introduced into the cells by any method known in the art, including but not limited to: transduction utilizing a viral vector; transfection, including chemical (e.g. liposome)-mediated transfection, electroporation, sonoporation, impalefection, optical transfection, and particle-based transfection (gene gun); and transformation, including chemical-mediated transformation and electroporation. One of skill in the art will recognize that the nucleic acids disclosed herein can be transferred to a β-cell to be administered to a subject, thereby resulting in upregulation or overexpression of Stx4, and thus a gene product, in the β-cell.

In certain embodiments, the introduction of nucleic acid(s) into the β-cells to be administered to a subject is by transduction via a viral vector. The viral vector can be any viral vector suitable for transduction, including but not limited to a retroviral vector, adenovirus, herpes simplex virus, lentivirus, poxvirus, adeno-associated virus, and recombinant adeno-associated virus (rAAV). In certain embodiments, the viral vector is recombinant adeno-associated virus.

The nucleic acid, whether naked DNA or incorporated into a vector, may optionally be operably linked to a promoter. The promoter can be chosen to affect transcription and/or translation of the nucleic acid, and is ideally functional in a desired host cell. The promoter can be β-cells specific, promoting transcription and/or translation only in β-cells. In certain aspects, it may desirable to prevent upregulation or overexpression of a gene outside of the β-cells in which upregulation or overexpression was initially induced.

The β-cells in which overexpression of Stx4 is to be induced can be autogenic, allogenic, or xenogenic, originating from the same subject on which the method is being performed, a different subject of the same species as that on which the method is being performed, and a subject of another species, respectively.

Methods for differentiating stem cells into β-cells, differentiating precursor cells into β-cells, and reprogramming other cells, such as pancreatic cells, into β-cells have been developed; see, for example, US20110280842, describing methods of reprogramming cells into β-cells, and WO2000078929, describing methods of dedifferentiating pancreatic cells and obtaining pancreatic islet cells from the dedifferentiated pancreatic cells.

β-cells can be isolated from a subject or donor, induced to overexpress Stx4 , and administered to the subject. Administration can be undertaken by any means suitable to introduce cells to a subject, such as transplantation.

Alternatively, β-cells overexpressing Stx4 can be transplanted at a targeted site where improved insulin secretion and/or sensitivity is desired. The β-cells overexpressing Stx4 can be transplanted in a target location or tissue, for example, pancreatic tissue, liver tissue, skeletal muscle tissue, adipose tissue, brain tissue, heart tissue, spleen tissue, kidney tissue, and lung tissue.

When isolating β-cells from the subject or a donor, separation or isolation of the β-cells from the connective matrix and remaining exocrine tissue is advantageous. A widely used method for transplanting β-cells is known as the “Edmonton Protocol.” The Edmonton Protocol transplants healthy β-cells into diabetic patients. β-cell transplantation using the Edmonton Protocol is described in Shapiro et al. Transplantation Proceedings, 33, pp. 3502-3503 (2001); Ryan et al., Diabetes, Vol. 50, April 2001, pp. 710-719; and Ryan et al., Diabetes, Vol. 51, July 2002, pp. 2148-2157. Once in the liver, the cells develop a blood supply and begin producing insulin. The Edmonton Protocol, or any other islet cell transplantation protocol, can be used to administer β-cells overexpressing Stx4.

In addition to administration of the β-cells overexpressing Stx4 , a cell therapy described herein can further comprise administration of anti-inflammatory and/or immunosuppressive drugs prior to, concurrently, or following the administration of the cells. Inflammatory response associated with islets is a primary cause of early damage to β-cells and graft loss after transplantation. Harmful inflammatory events can occur during pancreas procurement (from the subject or a donor), pancreas preservation, islet isolation, and islet infusion. Controlling pre- and peritransplant islet inflammation improves posttransplant islet survival, enhancing the benefits of the transplantation. Suitable drugs include but are not limited to cyclosporin, FK506, rapamycin, corticosteroids, cyclophospham ide, mycophenolate mofetil, leflunom ide, deoxyspergualin, azathioprine, OKT-3, and the like.

Due to the unexpected and improved protection against inflammatory damage in the β-cells overexpressing Stx4 , the effective amount of anti-inflammatory drugs administered may be lower than that administered with β-cells without Stx4 overexpression.

Immunosuppressive medications can be helpful in preventing rejection of the transplant. Use of anti-inflammatory and/or immunosuppressive drugs can also be beneficial during systemic administration of the β-cells overexpressing Stx4.

In certain embodiments of the cell therapy method disclosed herein, the β-cells are autologous β-cells isolated from a subject, transduced to induce overexpression of Stx4, and transplanted back into the subject.

### Gene Therapy

Wherein overexpression of Stx4 in the subject is induced by gene therapy, the gene therapy comprises administering to the subject at least one nucleic acid encoding Stx4.

Overexpression of Stx4 can be achieved in a subject by administering to the subject a nucleic acid having a sequence identity at least 80% identical to the human Stx4 gene or the human Stx4 gene with a L173A/E174A mutation.

In certain embodiments, the nucleic acid can have a sequence identity at least 80%, at least 85%, at least 90%, at least 95%, at least 96%, at least 97%, at least 98%, at least 99%, or 100% identical to the human Stx4 gene or the human Stx4 gene with a L173A/E174A mutation. The nucleic acids described herein, once administered to the subject, result in the upregulation or overexpression of Stx4.

The nucleic acids can be administered as naked DNA, or as part of a plasmid or vector. Where nucleic acids are present in a vector, the vector may be viral or non-viral. DNA- and RNA-liposome complex formations are examples of useful non-viral vectors. Such complexes comprise a mixture of lipids which bind to genetic material (DNA or RNA), providing a hydrophobic coat which allows the genetic material to be delivered into cells. Liposomes which can be used include DOPE (dioleyl phosphatidyl ethanol amine) and CUDMEDA (N-(5-cholestrum-3-˜-ol 3-urethanyl)-N′,N′dimethylethylene diamine).

When Stx4 is administered using a liposome, it is preferable to first determine in vitro the optimal DNA: lipid ratio and the absolute concentration of DNA and lipid as a function of cell death and transformation efficiency for the particular type of cell to be transformed. These values can then be used, or extrapolated for use, in vivo administration. The in vitro determination of these values can be readily carried out using techniques known in the art. Some other examples of non-viral vectors include non-lipid cationic polymers [polyethylenimine (PEI), polyamidoamine (PAMAM), poly-L-lysine], hemagglutinating virus of Japan-envelope (HVJ-E, an inactivated Sendai virus envelope), cationic liposomal lipid (Lipofectamine), and cationic non-liposomal lipids (Effectene), were developed for favorable transfection efficiency in gene transfer.

Other non-viral vectors can also be used in accordance with the present disclosure. These include chemical formulations of nucleic acids coupled to a carrier molecule or other molecule which facilitates delivery to target cells and tissues for the purpose of altering the biological properties of the host cells (e.g., increasing insulin secretion or sensitivity).

Exemplary protein carrier molecules include antibodies specific to the islet cells or receptor ligands, i.e., molecules and peptides capable of interacting with receptors associated with a cell of a targeted secretory gland.

Other methods for delivering nucleic acids to β-cells include, for example, lipoplex condensation and encapsulation, polymersome condensation and encapsulation, polyplex complex formation, dendrimer complex formation, inorganic nanoparticle complex formation, and cell penetrating peptide complex formation. These are further examples of possible chemical modifications of a nucleic acid described herein, facilitating delivery of the nucleic acid to a target cell or tissue. This delivery may be further facilitated by incorporating a β-cell specific peptide, thereby targeting the chemically modified nucleic acid particularly to β-cells.

Nucleic acids describe herein can also be administered to a subject in a viral vector. The viral vector can be a lentiviral vector, such as a human immunodeficiency virus (HIV) vector or a simian immunodeficiency virus (SIV) vector, an adenoviral vector or an AAV vector.

“HIV” is meant to include all clades and/or strains of human immunodeficiency virus 1 (HIV-I) and human immunodeficiency virus 2 (HIV-2). Likewise, the viral vectors can be self-inactivating (SIN) vectors, which have an inactivating deletion in the U3 region of the 3′ LTR. Such a deletion may include the deletion of the enhancer and/or promoter. SIN vectors are engineered so that transcription of the target gene can only be driven by an internal promoter once the expression cassette is integrated into the genome. Given the deletion in the U3 region, expression of Stx4 can be driven by a promoter. In certain embodiments, the gene transfer vector is selected from a retroviral vector, adenovirus, lentivirus, adeno-associated virus, recombinant adeno-associated virus, poxvirus, herpes simplex virus, and the like.

Self-inactivating retroviral vectors can also be used. The self-inactivation of the retroviral vector minimizes the risk that Replication Competent Retrovirus (RCRs) will emerge. It also reduces the likelihood that cellular coding sequences located adjacent to the vector integration site will be aberrantly expressed, either due to the promoter activity of the 3′ LTR or through an enhancer effect. Finally, a potential transcriptional interference between the LTR and the internal promoter (used for expression in tissue/cells of interest) driving the transgene is prevented by the SIN vector design.

In certain embodiments, the vector is an adeno-associated virus, due to the vector's minimal immunogenicity and high safety profile. Recombinant adeno-associated virus (rAAV) vectors appear to offer a vehicle for safe, long-term therapeutic gene transfer afforded through the propensity of rAAV to establish long-term latency without deleterious effects on the host cell and the relative non-immunogenicity of the virus or viral expressed transgenes.

The nucleic acids described herein, whether naked DNA, or incorporated into a plasmid or vector, can be administered to the subject by any means known in the art, including but not limited to intravenous injection, direct organ or tissue injection, organs surface instillation, intra-arterial injection, intraportal injection, and retrograde intravenous injection. These are all methods known in the art for gene delivery in a subject.

As described herein, Stx4 has either no effects or only positive effects when overexpressed in other tissue types, the nucleic acids can be administered systemically (e.g., intravenously).

To improve delivery of the nucleic acid, physical methods, including but not limited to electroporation can be used in conjunction with any particular method of administration. These physical methods have been shown in the art to enhance gene delivery by improving entry of the nucleic acid (whether or not in a vector) into cells.

Tissues beneficial for targeting by a method described herein include pancreatic tissue, skeletal muscle tissue, adipose tissue, brain tissue, heart tissue, liver tissue, spleen tissue, kidney tissue, and lung tissue.

Tissues or β-cells can be further targeted by operatively linking a nucleic acid described herein to a tissue and/or β cell specific promoter, which can limit expression of Stx4 to the target tissues and β-cells. Preferably the promoter is a strong, eukaryotic promoter. Exemplary eukaryotic promoters include promoters from cytomegalovirus (CMV), mouse mammary tumor virus (MMTV), Rous sarcoma virus (RSV), and adenovirus. More specifically, exemplary promoters include the promoter from the immediate early gene of human CMV and the promoter from the long terminal repeat (LTR) of RSV. Of these two promoters, the CMV promoter provides for higher levels of expression than the RSV promoter. There are a number of promoters known in the art that are capable of driving expression of Stx4.

Examples of pancreas specific promoters include the human insulin promoter and pancreas a-amylase promoters. Other beneficial promoters include for, example, skeletal muscle-specific promoters, kidney-specific promoters, and liver-specific promoters. The promoters can be derived from the genome of any mammal, and are preferably derived from a murine or a human source, more preferably from a human source.

The methods and materials disclosed herein can also be used to prolong the lifespan of a subject. By maintaining or improving β-cell life expectancies and protection from inflammatory damages, insulin secretion and/or sensitivity of a subject may be maintained or improved, and thereof the lifespan of a subject may be extended. In certain embodiments, the lifespan of a mouse is extended by a method described herein.

Pharmaceutical Compositions (Nucleic Acid)

Also described herein are pharmaceutical compositions comprising at least one nucleic acid encoding Stx4 operably linked to a promoter. As described above, the promoter is functional in eukaryotic cells, and is β-cell specific. Such compositions can be used in gene therapy methods described herein.

The pharmaceutical compositions disclosed herein can be administered to the subject by at least one method selected from the group consisting of: intravenous injection; direct organ or tissue injection; organ surface instillation; intra-arterial injection; intraportal injection; and retrograde intravenous injection. In some embodiments described herein, administration further comprises at least one physical method to enhance delivery of the nucleic acid selected from the group consisting of: electroporation; sonoporation; mechanical massage; and ultrasound exposure. In certain embodiments, the at least one physical method is applied to pancreatic tissue.

In certain embodiments of the pharmaceutical compositions disclosed herein, the pharmaceutical composition comprises at least one nucleic acid, wherein the at least one nucleic acid shares a sequence identity of at least 80% with at least one nucleic acid selected from the group consisting of the human Stx4 gene or the human Stx4 gene with a L173A/E174A mutation. In certain embodiments of the pharmaceutical compositions disclosed herein, the at least one nucleic acid shares a sequence identity selected from the group consisting of: at least 80%; at least 85%; at least 90%; at least 95%; at least 96%; at least 97%; at least 98%; at least 99%; and 100% with the at least one selected nucleic acid.

The at least one nucleic acid of the pharmaceutical composition can be present as naked DNA or in a vector. Suitable vectors are discussed above. In some embodiments, the vector comprises a sequence encoding a human insulin promoter and human Stx4 cDNA, wherein the human Stx4 cDNA is under the control of the human insulin promoter.

In certain embodiments, the at least one nucleic acid is chemically modified. The chemical modification is selected from the group consisting of: lipoplex condensation and encapsulation; polymersome condensation and encapsulation; polyplex complex formation; dendrimer complex formation; inorganic nanoparticle complex formation; and cell penetrating peptide complex formation. In certain embodiments, the chemical modification further comprises the addition of a tissue-specific peptide.

In certain embodiments, the at least one nucleic acid of the pharmaceutical composition is present in a vector. The vector can be selected from the group consisting of a retroviral vector; adenovirus; herpes simplex virus; lentivirus; poxvirus; adeno-associated virus; recombinant adeno-associated virus (rAAV); and naked plasmid DNA.

In certain embodiments of the pharmaceutical compositions disclosed herein, the pharmaceutical composition further comprises a pharmaceutically acceptable excipient. The excipient can be a natural or synthetic substance, and can act as a filler or diluents for the at least one nucleic acid, facilitating administration to the subject. The excipient can also facilitate nucleic acid uptake into a target cell, or otherwise enhance overexpression of Stx4.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

### Materials and Methods

Mice: All mouse studies were conducted as per the Guidelines and Assurances of the City of Hope IACUC. Stx4 heterozygous knockout mice were generated as previously described (13, 14). To generate the βTG-Stx4 mice, commercially available Rat insulin promoter (RIP)-rtTA mice (Jax# 008250, Bar Harbor, Me.), were bred to the custom generated TRE-STX4 mice. TRE-STX4 mice were initially made by B6 blastocyst injection. Rat Stx4 cDNA was subcloned into the 5′ BamHl and 3′ Clal sites downstream of the pTRE; 29 founder mice were obtained, and of these, 4 lines (#10, 9403, 9407 and 9410) were phenotyped to show similar glucose and insulin tolerances, and the #9410 line pursued for all remaining studies. These mice were crossed with RIP-rtTA mice (rat insulin promoter, RIP, induces β-cell selectivity; rtTA=tetracycline/doxycycline (Dox) inducible; the RIP-rtTA model was chosen because the glycemia matches that of the background C57BL6 with no alterations of glucose homeostasis (purchased from Jax labs #008250). βTG-Stx4 mice are heterozygous for each transgene. Mouse insulin promoter (MIP)-rtTA mice were obtained as a gift from Philip Scherer (UT Southwestern) and used as described (15, 16) in place of the RIP-rtTA where indicated in the text. βTG-Stx4 transgenic mice were provided with or without doxycycline in drinking water (1-2 mg/ml acidified water bottled in dark red bottles to protect from light) for at least 3 weeks prior to experiments to yield stabilized transgene expression.

Feeding studies: All C57B1/6J mice were fed a 22% from fat diet after weening from Picolab (product # 5058) until they were transferred into the experimental room at 8-10 weeks. Chow mice were housed on sanichip bedding in group of 3-5, and fed standard maintenance chow from PicoLab (Product #5053) with 13% of calories from fat starting at 12 weeks. In high fat feeding paradigm, 8-week old double transgenic Skm-STX4 mice were fed a custom made 45% kcal from fat diet ad libitum from research diets (Product #D01030108) for 10 weeks. Food intake and weight were measured twice a week. Insulin resistant mice were placed on 45% kcal from fat diet supplemented with 625 mg Doxycycline from research diets (Product # D17100202) for 5 weeks, while control mice were maintained on the high fat diet without doxycycline. Mice were housed individually on sanichip bedding, and food was measured and changed twice weekly while body weight was measured once a week.

Extracellular flux analysis (Seahorse): Flexor digitorum brevis (FDB) muscle was extracted from both legs of 24-week old high fat fed male mice (+/−Dox) and dissociated in 4 mg/ml collagenase from Sigma (cat # C-0130) for 2 hrs. After dissociation, muscle fibers were cleaned up and transferred into immersion media. 100 μl of myofibers were plated per well of a 24 well Agilent seahore eXF24 plate with 10 μl Matrigel in every well. The cells were left to settle and adhere overnight and then ran on the Agilent extracellular flux analysis machine (eXF24) where cells were treated with oligomycin (4 μM), FCCP (1 μM), Pyruvate (10 μM) and Rotenone/Antinomycin A (0.5 μM) at time intervals to assess mitochondrial function. Cells were lysed with 1% NP40 lysis buffer and normalised to protein content. Three independent experiments from 3 different mice were conducted for this study.

Antibodies and cytokines: Stx4 antibody for immunoblot for mouse islets was generated as described (17, 18). All other antibodies were purchased: STX4 (for human islets, Chemicon, Temecula Calif.); Tubulin (for immunoblots, Sigma, St. Louis, Mo.); Cleaved caspase-3 (for immunoblots), t-NF-κB, p-NF-κB, and TBP were all from (Cell Signaling Inc. Beverly, Mass.); Goat anti-rabbit-HRP and anti-mouse-HRP secondary antibodies (Bio-Rad, Hercules, Calif.). Enhanced chemiluminescence (ECL) and ECL Prime reagents were purchased from Thermo. For immunohistochemistry, antibodies against STX4, glucagon, and insulin were purchased from Chemicon, Abcam, and DAKO, respectively. Pro-inflammatory cytokines were purchased from (Prospec, East Brunswick, N.J.) as described (19).

Intraperitoneal glucose tolerance test (IPGTT): IPGTT was conducted in 4-6 month old mice fasted for 6 hours prior to experimentation (08:00-14:00 h) and housed individually. Following sample collection of fasted blood, male or female knockout or transgenic mice were injected intraperitoneally with freshly made D-glucose (10 or 20% stock solutions for dose of 1 g/kg or 2 g/kg body weight, respectively) for IPGTT analysis. Blood glucose levels were taken directly preceeding injection (0 min) and then at 15, 30, 60, 90 and 120 minutes post injection. 2 pl of blood (post injection) was taken from the mouse tail and diluted with 5 pl saline, then loaded into cuvettes and measured immediately using hemoque machine.

Insulin Tolerance Test (ITT): 4-6 month old mice were fasted for 6 hours prior to experimentation (08:00-14:00 h) and housed individually. For the ITT, the mice were injected intraperitoneally with Humulin R at 0.75 U/kg body weight (Eli Lilly and Company, Indianapolis IN). Blood glucose levels were taken from tail blood at directly preceeding injection and then at 15, 30, 45, 60 and 90 minutes post injection.

Multiple low dose streptozotocin (MLD-STZ) protocol: Male mice were subjected to IPGTT at 10-12 weeks of age, prior to initiation of the MLD-STZ protocol (20). Mice were injected across five consecutive days with STZ (35 mg/kg body weight). Five days later (10 days after first injection), STX4^{+/+} (Wt) and STX4^{+/+} mice were fasted for 6 h (08:00-14:00), sampled for fasting blood glucose, then injected intraperitoneally with glucose (1 g/kg body weight) and blood glucose sampled from the tail vein every 30 min using the Hemocue glucometer (Mission Viejo, Calif.). Male βTG-Stx4 mice were given doxycycline (2 mg/ml) in drinking water for 3 weeks prior to initiation of MLD-STZ injections. Twenty-four days after initiation of the MLD-STZ protocol, mice were fasted for 6 h (08:00-14:00), then injected intraperitoneally with glucose (2 g/kg body weight) to perform IPGTT.

Isolation, immunoblotting and secretion assays using transgenic mouse islets: Pancreatic mouse islets were isolated as previously described (21), from 10-14 wk old male or female mice, as noted in the description of the drawings. Islets cultured overnight were hand-picked into microcentrifuge tubes containing Krebs-Ringer bicarbonate buffer (10 mM HEPES pH 7.4, 134 mM NaCl, 5 mM NaHCO₃, 4.8 mM KCl, 1 mM CaCl₂, 1.2 mM MgSO₄, 1.2 mM KH₂PO₄) containing 2.8 mM glucose and 0.1% BSA for 1 hr, then buffer changed to either 16.7 mM or 2.8 mM for 1 hr longer. Supernatant was collected for quantitation of insulin release by radioimmunoassay (Millipore). Islets were subsequently harvested in 1% NP-40 lysis buffer for evaluation of total protein or insulin content, as previously described (22).

Immunoblot analysis: Mouse hindlimb that was extracted and flash frozen immediately in liquid nitrogen. The hindlimb was then lysed using NP40 lysis buffer with DTT. Samples were rotated at 4 degrees for 5 mins and spun down. Supernatant with the whole cell lysate (WCL) was taken and aliquoted into tubes to avoid freeze thaw protein degradation. The samples were run on hand cast 10% SDS page gels, running 20 ug protein/sample/lane. Samples were immunoblotted for STX4 (homemade antibody, 1:3000), Tubulin (Sigma #T5168, 1:5000), and DRP1 (abcam #ab56788 1:1000), in 1% BSA in TBST with sodium azide. Secondary antibody Goat Anti-Rabbit IgG (H L)-HRP Conjugate (Bio-Rad # 172-1019) or Goat Anti-Mouse IgG (H L)-HRP Conjugate (Bio-Rad # 172-1011). Chemiilluminescence was achieved using Bio-Rad chemidoc touch and regular ECL.

Islet immunofluorescent confocal imaging: Islets were immediately fixed in 4% paraformaldehyde for 25 min and permeabilized in 3% Triton X-100 for 3-4 h at room temperature. Fixed islets were blocked in 5% donkey serum (Sigma, St. Louis, Mo.) in 0.15% Triton X-100 overnight at 4° C. Islets were equilibrated at room temperature in antibody incubation buffer (0.2% Triton X-100/1% BSA/PBS) for 20 min, followed by an overnight incubation with mouse anti-insulin and guinea pig anti-glucagon (1:1000) and rabbit anti-Syntaxin 4 (1:500) reconstituted in antibody incubation buffer at 4° C. Islets were then washed 3 times with 0.2% Triton X-100/1% BSA/PBS for 30 min at room temperature, followed by incubation with anti-Texas Red, FITC or Cy5 secondary antibodies (1:1000) overnight at 4° C. Islets were then washed 3 times in PBS, overlayed with vectashield mounting medium and coverslips mounted for imaging analysis using the Zeiss 510 confocal microscope. Pancreata from βSTX4-dTg mice with or without doxycycline treatment were fixed 4% paraformaldehyde and embedded in paraffin. Slides were dewaxed, rehydrated and stained with guinea pig anti-insulin (1:500), mouse pig anti-glucagon (1:200) and rabbit anti-Syntaxin 4 (1:100) antibodies at 4° C. for overnight. Pancreas were washed and followed by incubation with Alexa 647, 568 or 405 conjugated secondary antibodies (1:500) for 1 h. Pancreata were washed and overlayed with vectashield mounting medium and coverslips mounted for imaging analysis using the Zeiss 700 confocal microscope.

TSE metabolic caging/body composition: All animal experiments were conducted in accordance with NIH Guide for the Care and Use of Laboratory Animals (National Institutes of Health Publication no. 85-23, revised 1996) and approved by the Institutional Animal Care and Use Committees of City of Hope National Medical Center (Duarte, Calif., USA). The metabolic cage system used for this experiment was the Phenomaster (TSE Systems, Bad Homburg, Germany). Mice were weighed, then individually housed in cages. Animals were given ad libitum access to food and water for the duration of the experiment. Animals were acclimatized to the cages for 1 day and experimental data was collected for 48 hours following the acclimation day. The light cycle was from 6:00 to 18:00 hours. Experimental parameters monitored included food intake, water consumption, oxygen intake, carbon dioxide production, and ambulatory activity. Animals were observed daily and end weights collected at the conclusion of the experiment.

Echo MRI: Whole body composition (fat and lean tissues) was determined using quantitative magnetic resonance technology (EchoMRI™ 3-in-1; Echo Medical Systems, Houston, Tex.). Automatic tuning and calibration of the instrument parameters using canola oil maintained at room temperature (22° C.) were performed daily. One mouse was placed in the analytical chamber. Parameters measured by the EchoMRI™ included lean mass (g), fat mass (g), free water (g), and total water (g).

Transmission Electron Microscopy: Mouse tissues were dissected and immediately fixed with 2.5% glutaraldehyte, 4% paraformaldehyde in 0.1M Cacodylate buffer (Na(CH₃)₂AsO₂·3H₂O), pH 7.2, at 4° C., overnight. Mouse tissues were washed three times with 0.1 M Cacodylate buffer, pH 7.2, post-fixed with 1% Os₄ in 0.1 M Cacodylate buffer for 30 minutes and then washed three times with 0.1 M Cacodylate buffer. The tissues were washed in H₂O three times, and stained in 1% uranyl acetate in H₂O at 4° C., overnight. The samples were washed in H₂O three times, and dehydrated through 30%, 50%, 60%, 70%, 80%, 95% ethanol, 100% absolute ethanol (twice), propylene oxide (twice), and were left in propylene oxide/Eponate (1:1) overnight at room temperature. The vials were sealed. The next day the vials were left open for 2-3 hours to evaporate propylene oxide. The samples were infiltrated with 100% Eponate and polymerized at ˜64° C. for 48 hours. Ultra-thin sections (˜70 nm thick) were cut using a Leica Ultra cut UCT ultramicrotome with a diamond knife, picked up on 200 mesh Formvar/carbon coated copper EM grids. Ultra-thin sections were stained with Reynold's lead citrate for 1 minute.

Immunogold staining (conducted by the Electron Microscopy core at the City of Hope): Mouse muscle tissues were dissected and immediately fixed with 0.2% glutaraldehyte, 4% paraformaldehyde in phosphate buffered saline (PBS), pH 7.4, at 4° C., overnight. Mouse tissues were then sectioned into 150 μm-thick sections using a Leica Vibratome. Vibratome sections were washed 3×5 minutes in 0.01M PBS, followed by incubation in freshly prepared 1% NaBH₄ in PBS to eliminate residual glutaraldehyde. Vibratome sections were washed 6×5 minutes in PBS, followed by incubation in blocking solution (normal goat serum diluted 1:10 in PBS) for 30 minutes and in rabbit anti-syntaxin 4 primary antibody (Chemicon) at 4° C., overnight. Vibratome sections were washed 3×5 minutes in PBS, and stained with Nanogold® anti-rabbit IgG (Nanoprobes) diluted 1:100 in PBS for 1.5 hours. Vibratome sections were washed 3 X 5 minutes in PBS and post-fixed with 1% glutaraldehyde in PBS for 10 minutes, and then washed 3×5 minutes in 1% BSA and 3×2 minutes in distilled water. HQ Silver enhancement kit (Nanoprobes) was used to further develop the Nanogold® staining for 3-6 minutes. Vibratome sections were washed 2×5 minutes in PBS, and post-fixed with 0.5% OsO₄ for 20 minutes and processed through steps of serial dehydration. Vibratome sections were embedded and polymerized in Eponate resin. Ultra-thin sections (˜70 nm thick) were cut using a Leica Ultra cut UCT ultramicrotome with a diamond knife, picked up on 200 mesh Formvar/carbon coated copper EM grids. Ultra-thin sections were stained 1% uranyl acetate and Reynold's lead citrate before imaging on an FEI Tecnai 12 transmission electron microscope equipped with a Gatan Ultrascan 2K CCD camera. Transmission electron microscopy was performed on an FEI Tecnai 12 transmission electron microscope equipped with a Gatan Ultrascan 2K CCD camera. Statistics. All data are presented as mean ±SEM. Students t-test for single comparisons or 2-way ANOVA were used for multiple comparisons. P less than 0.05 was considered significant.

Human islet transduction, subcellular fractionation, and immunoprecipitation: Human islets (obtained through the Integrated Islet Distribution Program, IIDP or City of Hope Islet Core, donor information listed in Table 1) were accepted for use if at least 80% pure with 75% viability.

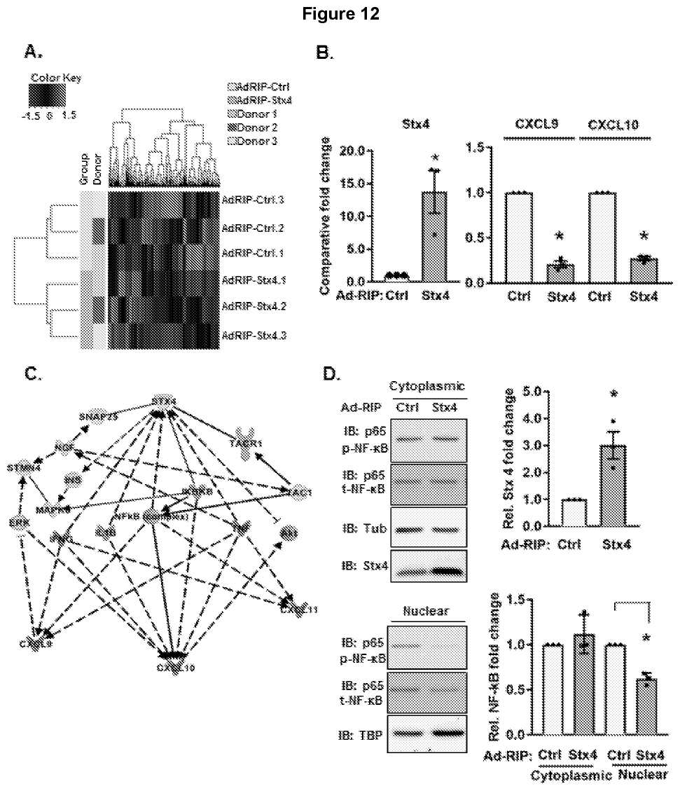

TABLE 1
Human Islet Donor Characteristics

| | | Age | | | | |
|---|---|---|---|---|---|---|
| Gender | Race | (year) | BMI | HbA1c | Purity | Experiment |
| M | Caucasian | 57y | 23 | 5.7 | 85% | RNA-seq, |
| | | | | | | qRT-PCR |
| F | Caucasian | 32y | 24.9 | 5.3 | 90% | RNA-seq, |
| | | | | | | qRT-PCR |
| M | Caucasian | 35y | 27.4 | 5.3 | NA | RNA-seq, |
| | | | | | | qRT-PCR |
| M | Hispanic | 15y | 24.5 | 5.1 | NA | qRT-PCR |
| F | Hispanic | 35y | 28.7 | 4.9 | NA | Western blot |
| | | | | | | of NE (Fig 12) |
| M | Asian | 37y | 29.5 | 5.3 | 70% | Western blot |
| | | | | | | of NE (Fig 12) |
| M | Hispanic | 49y | 28.9 | 5.1 | 80% | Western blot |
| | | | | | | of NE (Fig 12) |
| F | Caucasian | 32y | 39.4 | NA | 98% | Perifusion, |
| | | | | | | Cytokine treatment |
| M | African | 52y | 36.7 | NA | 90% | Perifusion, |
| | American | | | | | Cytokine treatment |
| M | Caucasian | 27y | 23.9 | NA | 80% | Cytokine treatment |
| F | Caucasian | 48y | 32.8 | 5.0 | 90% | Perifusion |
| F | Caucasian | 40y | 35.4 | NA | 90% | Perifusion |
| F | Caucasian | 45y | 27.4 | NA | 85% | Perifusion |
| M | Hispanic | 28y | 22 | 4.8 | 83% | IP with Stx4 |
| M | Caucasian | 46y | 33.2 | 5.5 | 85% | IP with Stx4 |
| M | Hispanic | 38y | 25.2 | 5.4 | 83% | IP with Stx4 |
| M | Caucasian | 37y | 29.5 | 5.5 | 75% | IP with Stx4 |
| M | Hispanic | 26y | 25.4 | 5.2 | 75% | IP with Stx4 |

NA; not applicable (not reported for non-diabetic donors),
IP: Immunoprecipitation,
NE: nuclear extract.

Upon receipt, human islets were first allowed to recover in CMRL medium for 2 h, and then were handpicked under a light microscope equipped with a green gelatin filter to discriminate residual non-islet material. Rat Stx4 cDNA was subcloned into the 5′ EcoRI and 3′ BamHI sites of the FN611 adenoviral vector, with the FN611 vector (23) containing the Ins2/RIP promoter to drive expression of STX4 in a beta cell-selective manner. The empty FN611 vector was used as the control. Adenoviruses were packaged with green fluorescent protein (RSV-GFPin the E3 region) to facilitate identification of transduced cells. Cesium chloride-purified adenoviral particles were generated (Viraquest, North Liberty, Iowa). Islets of non-diabetic donors were transduced at MOI of 100 with FN611 (RIP-Ctrl) or (RIP-STX4) adenoviral particles for 1 h at 37° C. Transduced islets were then washed twice and incubated for 48 h in medium at 37° C., 5% CO_{2.} In the last 16 h, islets were incubated in medium containing pro-inflammatory cytokines (TNFα; 10 ng/mL, IFNγ; 100 ng/mL, and IL-1β: 5 ng/mL), as previously described (19). Detergent cell lysates were prepared by harvesting in 1% Nonidet P-40 lysis buffer (1% NP-40, 25 mM HEPES pH 7.4, 10% glycerol, 50 μM sodium fluoride, 10 mM sodium pyrophosphate, 1 mM sodium vanadate, 137 mM sodium chloride, 1 mM phenylmethylsulfonyl fluoride, 1 μg/ml pepstatin and 10 μg/ml aprotinin) and lysates cleared by microcentrifugation for 10 min at 4° C. for use in immunoblotting. Human islets transduced with RIP-Ctrl or RIP-Stx4 were partitioned into nuclear and cytoplasmic fractions using the NE-PER kit (Thermo). Fractions were used for immunoblot analyses for localization of t-NF-kB and p-NF-kB using the NF-kB pathway kit (Cell signaling).

EndoC-βH1 and MIN6 cell culture: EndoC-βH1 cells obtained from Dr. Roland Stein (Vanderbilt University) were cultured as described previously (24). Adenovirus transduced at MOI=50-100 for 2 hr, washed with PBS and incubated in EndoC-βH1 medium for 48 h. Cells were then incubated for 16 h in medium containing pro-inflammatory cytokines (18). MIN6 p-cells were cultured as described previously (25) and transduced at MOI=50-100 for 2 hr, washed with PBS and incubated in MIN6 medium for 48 h. Cells were then incubated in medium containing pro-inflammatory cytokines (18) for the hours noted in the figures, and cleared cell lysates prepared for immunoblotting as described above with islets.

TUNEL staining and morphometric assessment of islet cell mass: Pancreatic sections were immunostained with rabbit anti-insulin (1:200 dilution, Santa Cruz Biotechnology, Dallas, Tex., USA). Alexa Fluor 488 goat anti-rabbit IGG (H+L) (1:500 dilution, Invitrogen) secondary antibody was used for detection of insulin. The TUNEL in Situ Fluorescein Cell Death Detection Kit (Roche, Mannheim, Germany) was used to stain apoptotic cells. Sections were scanned using a Keyence microscope (Keyence Itasca Ill.). Results were expressed as the percentage of cells positive for TUNEL staining relative to the total number of insulin-positive cells. Mouse islet morphometry was evaluated using anti-insulin immunohistochemical staining of pancreatic sections as described (22).

Human islet RNA isolation, RNAseq and quantitative real-time PCR: RNA was isolated from 3 Non-diabetic donors using QIAGEN RNeasy plus mini kit and processed with Illumina Truseq mRNA Library Prep kit. The RNA integrity number (RIN) values for all samples were 8.0 and above. Human islets were infected 100 MOI of beta cell specific expressed Stx4 or Control adenovirus for 1 hr, washed with PBS and incubated with CMRL1066, 10% FBS and 1% PSG for up to 48h. For confirming RNA sequencing data, qRT-PCR were performed. Fifty nanograms of RNA were directly used for quantitative real-time PCR by one-step qRT-PCR kit (Qiagen) with SYBR. Primers used to confirm RNA sequencing are listed in Table 2.

TABLE 2
Primers used for qRT-PCR

| Gene | | Sequence |
|---|---|---|
| STMN4 | Forward | 5′-ATCCTGAAGCCACCCTCCTTTG-3′ |
| | Reverse | 5′-TCCCTGTTCTCCTTGTTGGATTC-3′ |
| CECAM5 | Forward | 5′-AGCCTCACTTCTAACCTTCTGGAAC-3′ |
| | Reverse | 5′-GTAGCCAAAAAGATGCTGGGG-3′ |
| CXCL9 | Forward | 5′-ACTATCCACCTACAATCCTTGAAAGAC-3′ |
| | Reverse | 5′-TCACATCTGAATCTGGGTTTAG-3′ |
| CXCL10 | Forward | 5′-AGCAAGGAAAGGTCTAAAAGATCTCC-3′ |
| | Reverse | 5′-GGCTTGACATATACTCCATGTAGGG-3′ |
| Stx4 | | QT00014679 |
| Tubulin | | QT00089775 |

Human tubulin (Qiagen: QT00089775) was used for normalizing control. qRT-PCR conditions: cDNA synthesis at 50° C. for 30 min and 95° C. for 2 min hold, then 40 cycles of 95° C. for 15 sec, 58° C. for 30 sec and 72° C. 15 sec. Reads were aligned against hg19 using TopHat2. Read counts were tabulated using htseq-count, with UCSC known gene annotations (TxDb.Hsapiens.UCSC. hg19.knownGene). Fold-change values were calculated from Reads Per Kilobase per Million reads (RPKM,) and normalized expression values, which were also used for visualization (following a log2 transformation). P-values were calculated from raw counts using edgeR, and false discovery rate (FDR) values were calculated using the method of Benjamini and Hochberg. Prior to p-value calculation, genes were filtered only to include transcripts with a RPKM expression level of 0.1 in at least 50% of samples as well as genes that are greater than 150 bp. Genes were defined as differentially expressed if they had a fold-chang|>1.5 and FDR<0.05. Gene Ontology (GO) enrichment was calculated using goseq. Additional systems-level analysis was performed in IPA (Ingenuity® Systems, www.ingenuity.com).

Co-immunoprecipitation: Human EndoC-βH1 cells were transduced with Ad-RIP-Stx4 or Ad-RIP Ctrl as described above, then were harvested in 1% NP40 lysis buffer. Immunoprecipitation reactions (IP) included cleared lysate (1 mg) proteins plus either anti-Stx4 or anti-NF-kB antibodies (1 μg), incubated overnight at 4° C., followed by a second incubation with Protein G Plus-agarose for 2 h. Immunoprecipitates were washed in lysis buffer, boiled in Laamli sample buffer, and proteins resolved on 10% or 12% SDS-PAGE followed by transfer to PVDF membranes for immunoblotting.

Quantification and Statistical Analysis: All data were evaluated for statistical significance using Student's t test for pairwise comparison of two groups (i.e. STX4^{+/-} versus STX4^{+/+}) or ANOVA for comparisons of 3 or more groups, using GraphPad Prism. Data are expressed as the mean ±S. E., and judged to be statistically significant when p<0.05.

### Example 1: Association between Stx4 Deficiency and Increased Susceptibility for Non-Obese Diabetes

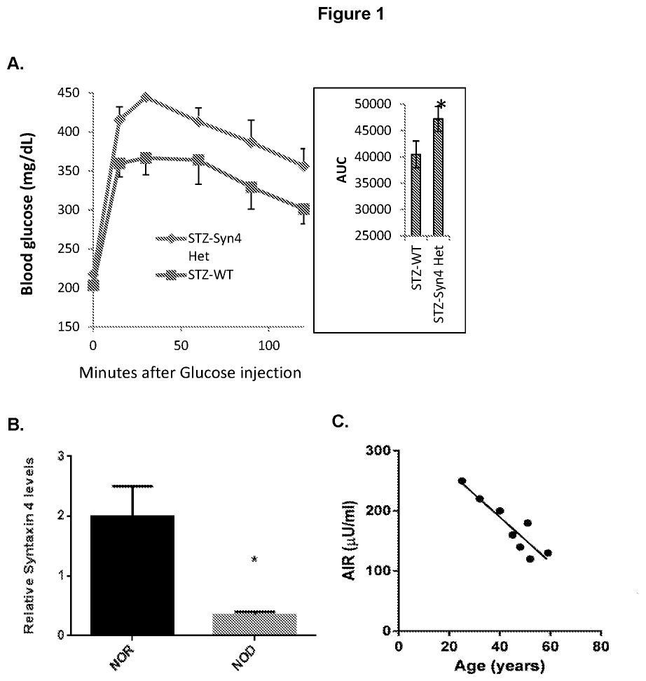

Stx4 heterozygous knockout (Syn4-het) mice were subjected to multiple low dose streptozotocin (MLD-STZ) treatment. Syn4-het mice treated with MLD-STZ were more severely glucose intolerant than wild-type control mice (FIG. 1A). By extrapolation, individuals with genetic susceptibility (SNPs for Stx4), or subjected to environmental conditions that result in Stx4 deficiency, may be more at-risk of developing T1D. In addition, 8 NOD and 8 NOR mice at 13 weeks of age were examined for their Stx4 protein levels in islets, as a precursor of pre-type 1 diabetic susceptibility. FIG. 1B shows that islets from NOD mice at this age were ˜50% deficient in Stx4 protein. A correlation between the increasing age and decreased Stx4 protein content in pancreas was previously reported (22), and islets from older human donors showed reduced function. The 1st phase GSIS and human islet donor age showed astatistically significant inverse association (FIG. 1C, R²=0.84, p=0.0014). Collectively, these data suggest that a partial depletion of Stx4 may correlate with an increased susceptibility to inflammatory damage to beta cells, in vivo.

### Example 2: N-Cell Selective Overexpression of Stx4 is Sufficient to Boost Glucose Tolerance

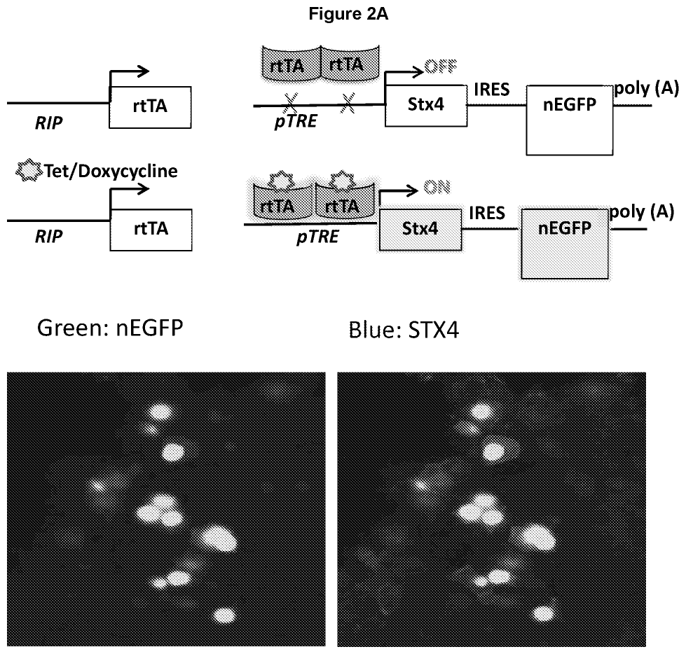

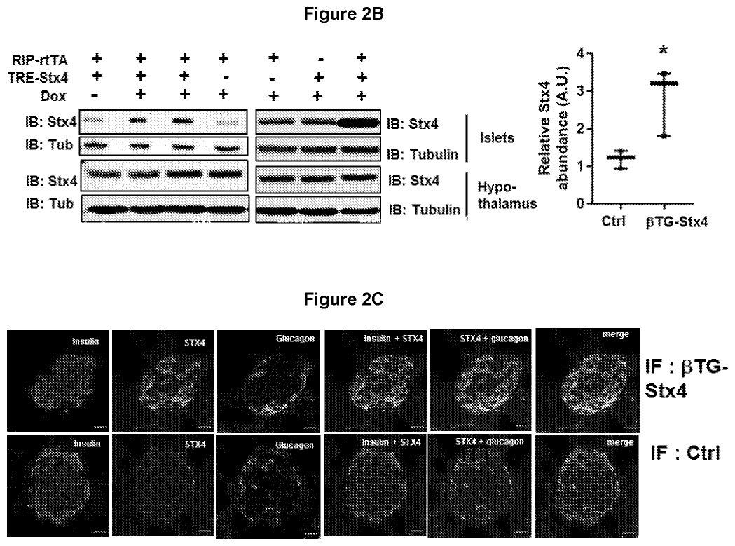

To study the impact of Stx4 upregulation selectively in the β-cell of an adult mouse, a doxycycline (dox)-inducible double transgenic model was used; the TRE-Stx4 transgene expression is driven by the dox-induced expression of the rat or mouse insulin promoter (RIP)-rtTA transgene, to confer β-cell selective expression of Stx4 (FIG. 2A, top). This design allays any concerns over developmental changes that may occur with transgene expression. Imaging of nEGFP (green in FIG. 2A, bottom left) and STX4 (blue in FIG. 2A, bottom right) in Dox stimulated TRE/RIP mice confirmed the dox-induced upregulation of Stx4 as designed.

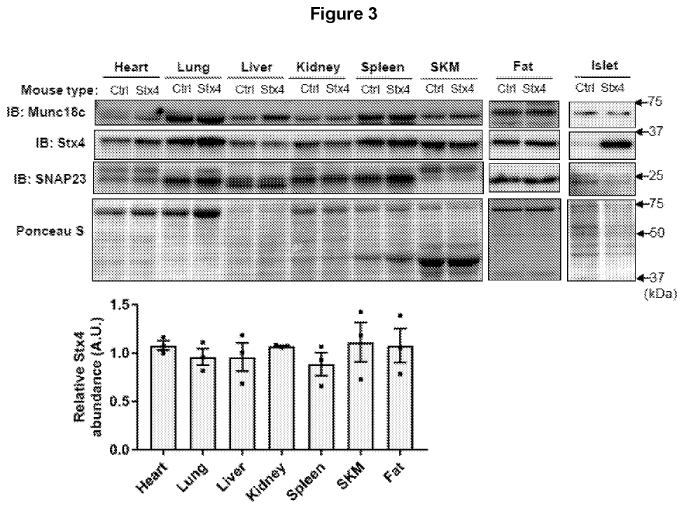

In this model, Stx4 expression is increased by ˜2-3-fold over endogenous levels in islets of double transgenic (referred to as βTG-Stx4) mice in response to Dox (FIG. 2B). As per design of the model, the dox-induced double transgenic (βTG-Stx4) mice did not over-express the Stx4 transgene in α-cells or hypothalamus, showing levels similar to control (Ctrl) non-dox-induced double transgenic or the RIP-rtTA single transgenic mouse lines (FIGS. 2B-2C). Tissues such as heart, lung, liver, kidney, spleen, skeletal muscle and epigonadal fat showed no changes to Stx4 abundance, nor are levels of its binding partners changed (FIG. 3).

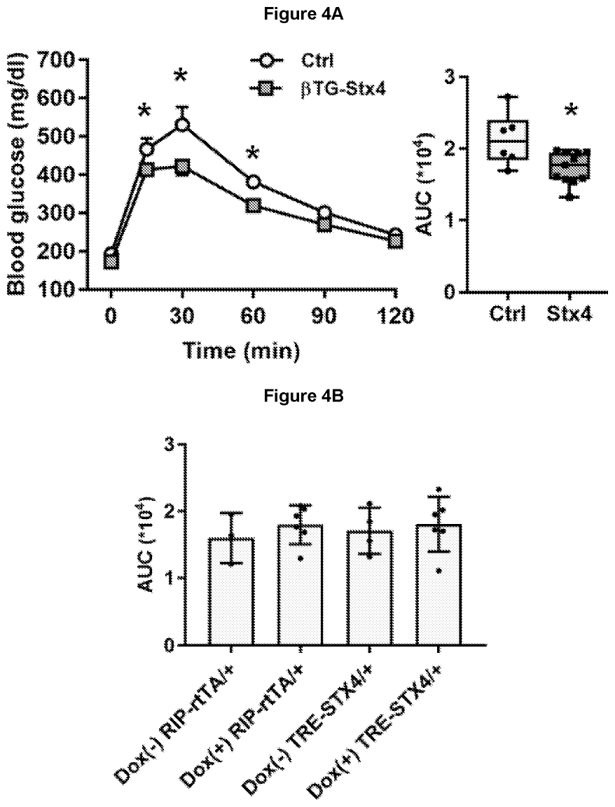

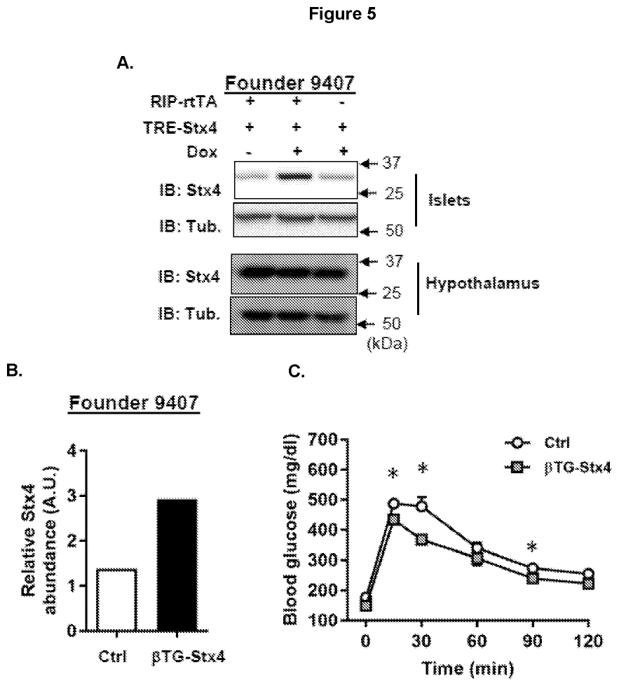

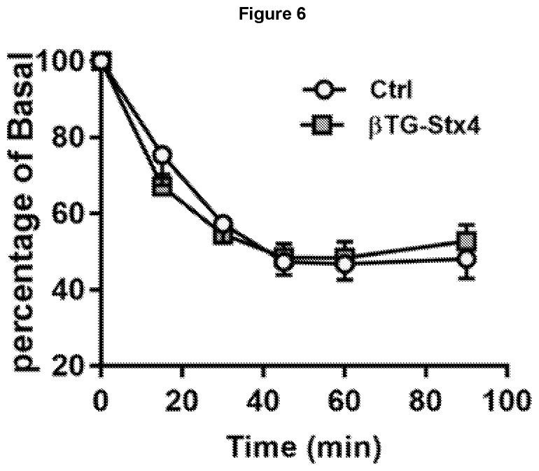

Dox-induced female βTG-Stx4 mice exhibit enhanced glucose tolerance in IPGTT assays compared with non-dox-induced double Tg mice (Ctrl) (FIG. 4A), or with single transgenic mice treated with or without dox (FIG. 4B). This pattern of transgene expression and physiological response was fully recapitulated in a second founder line (FIGS. 5A-5C). Dox-induced βTG-Stx4 female mice showed no change in insulin tolerance when compared with non-dox-treated controls (FIG. 6).

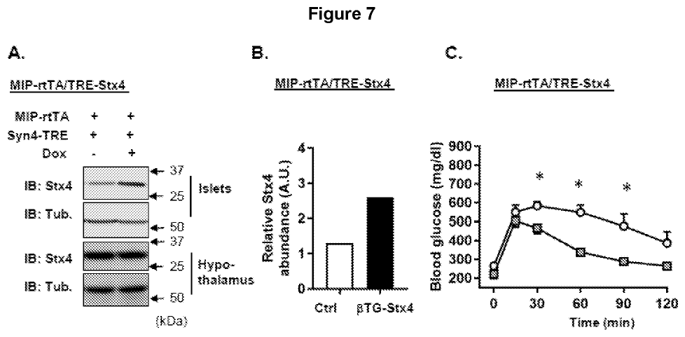

MIP-rtTA-based βTG-Stx4 mice served as a second model to study β-cell specific Stx4 overexpression in vivo; MIP-based βTG-Stx4 female mice phenocopied the RIP-based βTG-Stx4 mice (FIGS. 7A-C). These data indicate that the upregulation of Stx4 strictly in the 8-cell can promote enhanced glucose tolerance.

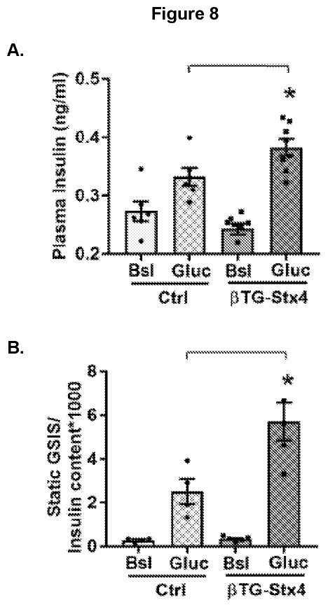

Serum insulin content was measured during the IPGTT for assessment of the magnitude of the in vivo acute response to the glucose stimulus. Dox-induced βTG-Stx4 mice elicited a significant increase in serum insulin content within 10 min of glucose injection, compared with non-dox induced mice (FIG. 8A). Importantly, basal insulin release in the dox-induced βTG-Stx4 mice was similar to that of control mice, indicating that the “boosting” effect of Stx4 upon function retains normal glucose responsiveness and does not cause aberrant elevation of insulin release under basal conditions. Islets isolated from the βTG-Stx4 mice showed a greater than >2-fold potentiation of GSIS in static islet culture assays (FIG. 8B). Notably, this level of enhancement is proportional to the ˜2-3-fold increased abundance of Stx4 protein in the islets. No differences in serum triglycerides, cholesterol, non-esterified fatty acids (NEFAs), or glucagon levels were found between Ctrl and βTG-Stx4 mice that were fasted for 16 h or for 6 h (Table 3).

TABLE 3
Fasting serum analytes of βrTG-STX4
mice compared with control littermates

| | 16 h fasting | | 6 h fasting | |
|---|---|---|---|---|
| | βTG-STX4 | Control | βTG-STX4 | Control |
| Triglycerides (mg/dl) | 28.0 ± 2.57 | 28.2 ± 1.88 | 29.6 ± 1.92 | 33.2 ± 2.48 |
| Cholesterol (mg/dl) | 55.0 ± 3.74 | 58.2 ± 3.40 | 54.7 ± 5.00 | 61.0 ± 2.48 |
| NEFA (mmol/L) | 0.88 ± 0.04 | 0.92 ± 0.04 | 0.41 ± 0.03 | 0.45 ± 0.04 |
| Glucagon (pg/ml) | 83.2 ± 8.86 | 74.0 ± 3.00 | 56.1 ± 5.50 | 54.2 ± 9.80 |

Data represent the average ± SE; no significant differences were detected. Serum was collected from 16 h fasted or 6 h fasted dox-induced βTG-STX4 and non-dox induced RIP-based double transgenic (Control) female littermate mice at 4-6 months of age (n = 5 for 16 h fasting, n = 6-7 for 6 h fasting per each genotype) for determination of parameters shown.

Similarly, no differences in body weights or organ/tissue weights were detected (Table 4).

TABLE 4
Body and tissue weights of βTG-STX4
mice compared with control littermates

| | % of body weight | | (g, gram) | |
|---|---|---|---|---|
| | βTG-STX4 | Control | βTG-STX4 | Control |
| Heart | 0.50 ± 0.09 | 0.46 ± 0.05 | 0.14 ± 0.02 | 0.13 ± 0.01 |
| Lung | 0.96 ± 0.10 | 0.75 ± 0.03 | 0.23 ± 0.03 | 0.20 ± 0.01 |
| Liver | 4.42 ± 0.18 | 4.40 ± 0.21 | 1.06 ± 0.03 | 1.15 ± 0.06 |
| Spleen | 0.47 ± 0.07 | 0.41 ± 0.10 | 0.11 ± 0.02 | 0.13 ± 0.03 |
| Kidney | 1.33 ± 0.07 | 1.29 ± 0.05 | 0.32 ± 0.02 | 0.32 ± 0.01 |
| Fat | 2.01 ± 0.47 | 1.92 ± 0.34 | 0.48 ± 0.10 | 0.52 ± 0.10 |
| Muscle | 1.32 ± 0.14 | 1.10 ± 0.07 | 0.31 ± 0.03 | 0.29 ± 0.02 |
| Pancreas | 0.73 ± 0.03 | 0.74 ± 0.02 | 0.18 ± 0.01 | 0.17 ± 0.01 |

Data represent the average ± SE; Weights were collected from dox-induced βTG-STX4 and non-dox induced RIP-based double transgenic (Control) female littermate mice at 4-6 months of age (n = 4-10) for determination of parameters shown. Body weight: βTG- STX4 (g): 24.05 ± 0.81, Control (g): 26.03 ± 0.69. No statistical differences were detected.

Taken together, the studies indicate that the positive effect of Stx4 upon islet function is accounted for by its effect upon the β-cell. These data also indicate an increased secretory capacity in the mice selectively over-expressing Stx4 in the β-cells.

### Example 3: Beta Cell-Specific Stx4-Over-Expressing Mice Resist STZ-Induced Diabetes

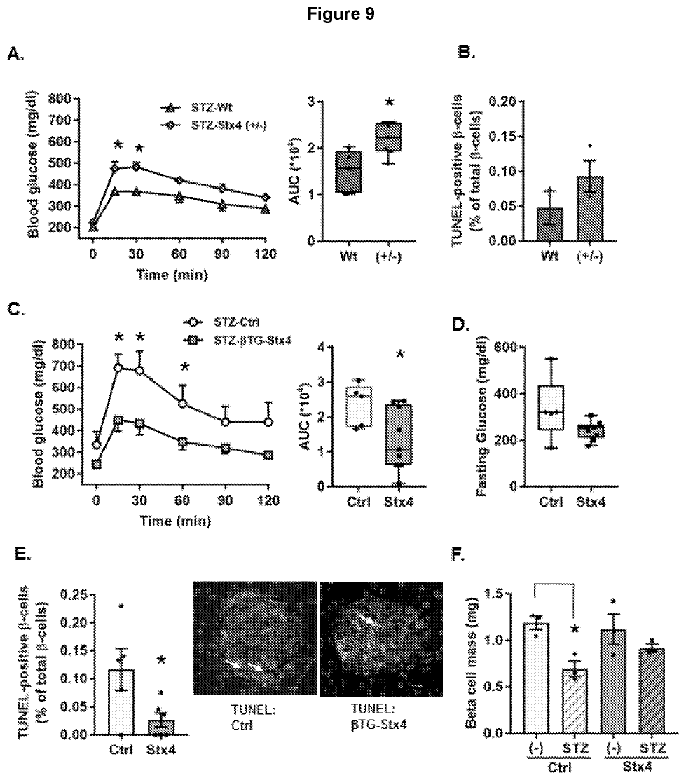

To test the working hypothesis that Stx4 abundance is crucial to the defense against proinflammatory-induced diabetes, male Stx4 heterozygous (Stx4^{+/-}) knockout mice were subjected to multiple-low-dose streptozotocin (MLD-STZ) treatment. FIG. 9A shows that Stx4^{+/-} mice treated with MLD-STZ were more severely glucose intolerant than wild-type (Wt) control mice within 10 d of the protocol initiation, concurrent with a trend for elevated levels of TUNEL+ β-cells (FIG. 9B). These data suggest that a partial depletion of Stx4 may correlate with an increased susceptibility to inflammatory damage to beta cells, in vivo.

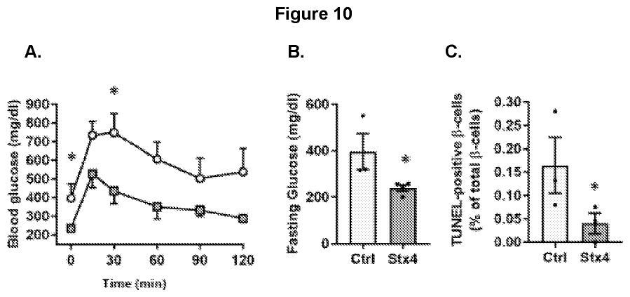

To determine whether the selective increase of Stx4 in the β-cells of the mice could offer protection from inflammatory damage associated with diabetes, dox-induced βTG-Stx4 male mice were challenged with MLD-STZ in parallel with non-dox-treated double transgenic mice (Ctrl). At 24 days following initiation of the MLD-STZ protocol, Ctrl mice exhibited severe glucose intolerance, whereas Dox-treated βTG-Stx4 mice retained a more normal level of glucose tolerance and fasting blood glucose (FIGS. 9C-9D). Pancreata from MLD-STZ-treated dox-induced βTG-Stx4 mice show reduced TUNEL+ β-cells and preserved β-cell mass, compared with MLD-STZ-treated Ctrl mice (FIGS. 9E-9F). MIP-based βTG-Stx4 mice also showed protection from the MLD-STZ-induced severe fasting hyperglycemia (>400 mg/dl) and glucose intolerance observed in the paired Ctrl mice (FIGS. 10A-10C).

These data indicate that Stx4 enrichment in the β-cell, provided to the adult mouse rather than during development, was sufficient to confer protection against diabetogenic stimuli targeting the islet β-cells.

### Example 4: β-Cell-Specific Stx4-Enrichment Protects Human Islets Against Pro-Inflammatory Cytokine Induced Apoptosis

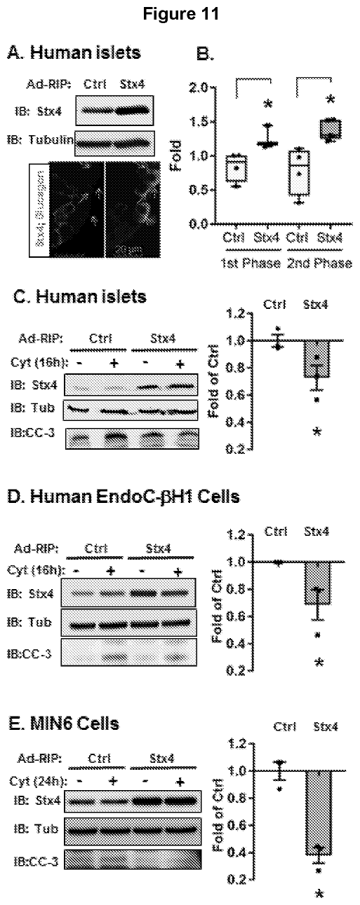

To investigate the effects of Stx4 selectively in the β-cells of human islets, an adenovirus driving Stx4 expression was generated using the RIP promoter. The virus successfully drove a ˜2-3 fold increase in Stx4 expression, and expression was not observed in glucagon-stained α-cells (FIG. 11A). Human islets harboring the extra Stx4 showed increased function in each phase of glucose-stimulated insulin secretion (GSIS) (FIG. 11B). Pro-inflammatory cytokine exposure induced apoptosis in Ad-RIP-Ctrl-transduced human islets, as determined by the presence of the cleaved caspase-3 fragment (CC-3) (FIG. 11C); human islets harboring Ad-RIP-Stx4 enrichment in the β-cells showed a substantial decrease in CC-3 expression. The reduction in cleaved caspase-3 (CC-3) expression positively correlated with the level of Stx4 over-expression in pure β-cell populations as well, both the human EndoC-βH1 and the mouse MIN6 β-cell lines (FIGS. 11D-11E). This is the first demonstration of a t-SNARE protein such as Stx4 acting as an anti-apoptotic factor.

### Example 5: RNAseq Analysis of Human Islets Specifically Over-Expressing Stx4 in β-Cells

To determine the potential contributing factors underlying the protection of Stx4-over-expressing β-cells from apoptosis, human islets transduced with Ad-RIP-Stx4 were compared with Ad-RIP-Ctrl transduced islets for differential gene expression using RNAseq. Three independent sets of donor human islets, each transduced with Ad-RIP-Ctrl or -Stx4, showed clear clustering by virus-type alone (FIG. 12A). Differential gene expression analyses pointed to endocrine systems, metabolism, and immunological diseases (Table 4). Ingenuity canonical pathways included largely immune pathways, showing the downregulation of genes such as CXCL9, CXCL10 and CXCL11, which are implicated in apoptosis and islet inflammation, coordinate with the increased Stx4 (Table 5).

TABLE 5
Diabetes related gene change and Ingenuity canonical pathways

| | Diseases or | | |
|---|---|---|---|
| | Functions | | |
| Categories | Annotation | p-value | Molecules |
| Endocrine System | insulin- | 1.18E−04 | CCL5, CD79B, |
| Disorders, Gastro- | dependent | | CR2, CXCL10, |
| intestinal Disease, | diabetes | | HLA-DOA, IKZF3, |
| Immunological Disease, | mellitus | | LILRB4, MUC21, |
| Metabolic Disease, | | | TRIM31, VIP |
| Organismal Injury and | | | |
| Abnormalities | | | |
| Metabolic Disease | glucose | 5.90E−04 | CCL5, CD79B, CP, |
| | metabolism | | CR2, CXCL10, |
| | disorder | | FGF19, GABRP, |
| | | | GNMT, HLA-DOA, |
| | | | IKZF3, LILRB4, |
| | | | MMP9, MUC21, |
| | | | NTS, PTF1A, STX4, |
| | | | TRIM31, VIP |
| Endocrine System | diabetes | 1.32E−03 | CCL5, CD79B, CP, |
| Disorders, Gastro- | mellitus | | CR2, CXCL10, |
| intestinal Disease, | | | FGF19, GABRP, |
| Metabolic Disease, | | | HLA-DOA, IKZF3, |
| Organismal Injury and | | | LILRB4, MMP9, |
| Abnormalities | | | MUC21, PTF1A, |
| | | | TRIM31, VIP |
| Ingenuity Canonical | | | |
| Pathways | p-value | Ratio | Molecules |
| Pathogenesis of Multiple | 8.13E−08 | 0.44 | CXCL10, CXCL11 |
| Sclerosis | | | CCL5, CXCL9 |
| B Cell Development | 0.0002 | 0.13 | HLA-DOA, CD79B, |
| | | | CD79A |
| IL-17A Signaling in | 0.0003 | 0.13 | CXCL10, CXCL11, |
| Gastric Cells | | | CCL5 |
| Granulocyte Adhesion | 0.0007 | 0.04 | CXCL10, CXCL11, |
| and Diapedesis | | | CCL5, CXCL9, |
| | | | MMP9 |
| Agranulocyte Adhesion | 0.0009 | 0.04 | CXCL10, CXCL11, |
| and Diapedesis | | | CCL5, CXCL9, |
| | | | MMP9 |
| Neuroprotective Role of | 0.0009 | 0.08 | NTS, TAC1, |
| THOP1 in Alzheimer's | | | MMP9 |
| Disease | | | |
| Altered T Cell and B Cell | 0.0037 | 0.05 | HLA-DOA, CD79B, |
| Signaling in Rheumatoid | | | CD79A |
| Arthritis | | | |
| Role of Hypercyto- | 0.0052 | 0.10 | CXCL10, CCL5 |
| kinemia/hyperchemo- | | | |
| kinemia in the Patho- | | | |
| genesis of Influenza | | | |
| Complement System | 0.0117 | 0.06 | C4BPB, CR2 |
| Lactose Degradation III | 0.0204 | 0.25 | GBA3 |
| PI3K Signaling in B | 0.0309 | 0.02 | CD79B, CD79A, |
| Lymphocytes | | | CR2 |
| Tryptophan Degradation | 0.0309 | 0.17 | IDO1 |
| to 2-amino-3-carboxy | | | |
| muconate Semialdehyde | | | |
| FcyRIIB Signaling in B | 0.0309 | 0.04 | CD79B, CD79A |
| Lymphocytes | | | |
| Communication between | 0.0309 | 0.04 | CXCL10, CCL5 |
| Innate and Adaptive | | | |
| Immune Cells | | | |
| Role of MAPK Signaling | 0.0398 | 0.03 | CXCL10, CCL5 |
| in the Pathogenesis of | | | |
| Influenza | | | |
| Airway Pathology in | 0.0407 | 0.13 | MMP9 |
| Chronic Obstructive | | | |
| Pulmonary Disease | | | |

The ratio is calculated that the number of molecules in a given pathway that meet cutoff |1.5|, divided by total number of molecules that make up that pathway that are in the reference set. Stx4 indicates increased expression; all others indicated decreased gene expression.

These RNAseq results were confirmed by quantitative PCR (FIG. 12B). In fact, CXCL10 has been identified as a dominant chemokine ligand expressed in the islet environment of T1D humans (26). Insulitic lesions of T1D patients but not non-diabetic pancreas donors showed expression of CXCL10, while islet infiltrating leukocytes expressed its receptor, CXCR3 (27). Intervention in CXCL10/CXCR3 chemotaxis prevented autoimmune diabetes in mice (28, 29).

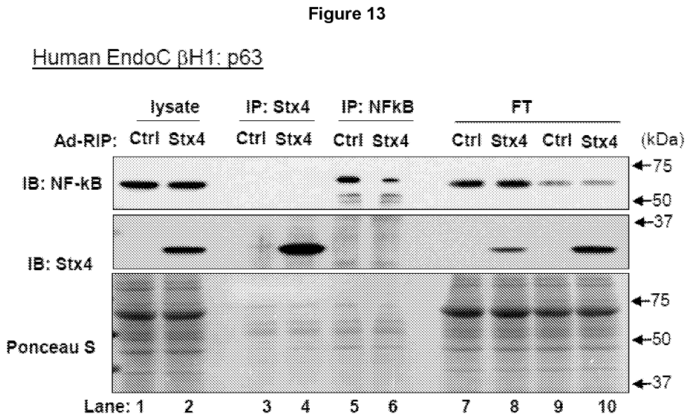

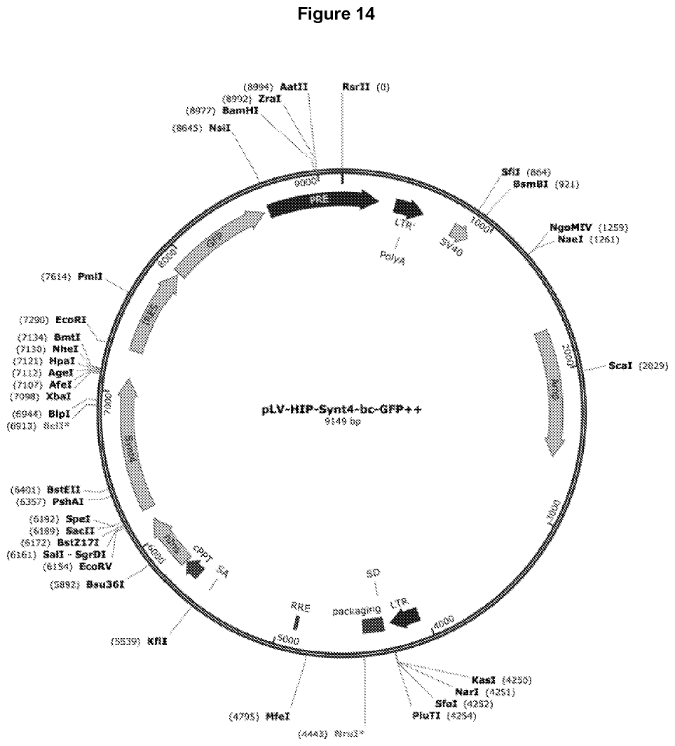

Network analysis was used to model potential linkages amongst the hits in the differential expression dataset, revealing a potential association between Stx4 and NF-κB (FIG. 12C). Indeed, phosphorylated NF-κB is known to translocate to the nuclei and transactivate promoters for CXCL9 and CXCL10 of stressed islet β-cells (26, 30). For example, islets exposed to metabolic, oxidative, or inflammatory stressors show p38 and c-Jun N-terminal kinase (JNK) mitogen-activated protein kinase (MAPK) signaling that promotes NF-κB to upregulate genes that promote apoptosis (31, 32). To test this as a potential mechanism by which Stx4 protects β-cells, human islets transduced with Ad-RIP-Ctrl or -Stx4 were subfractionated to partition cytoplasmic from nuclear proteins, and the locale of total (t) and phosphorylated (p)-NF-κB determined by immunoblotting. FIG. 12D shows a ˜40% decrease of p-NF-κB present in nuclear fractions made from Stx4-overeexpressing human islets. This is not via a direct association of Stx4 with NF-κB in the β-cells (FIG. 13). Moreover, RNAseq did not indicate changes in ER stress related genes such as ATF4, EIF2AK3; targeted mass spectrometry confirmed these findings (data not shown). These studies add a new twist to the effects of stress on β-cells in their own demise in T1D (33, 34). In addition to the production of the unfolded protein response (UPR) and neoantigens that may provoke autoimmune responses against β-cells, described herein is a defense mechanism of β-cells, in which Stx4 expression endorses protection from inflammatory and metabolic stress induced β-cell destruction and preservation of functional β-cell mass.

### Example 6: Selective Syntaxin 4 (STX4) Enrichment In Skeletal Muscle is Sufficient to Recapitulate Global Overexpression Phenotype

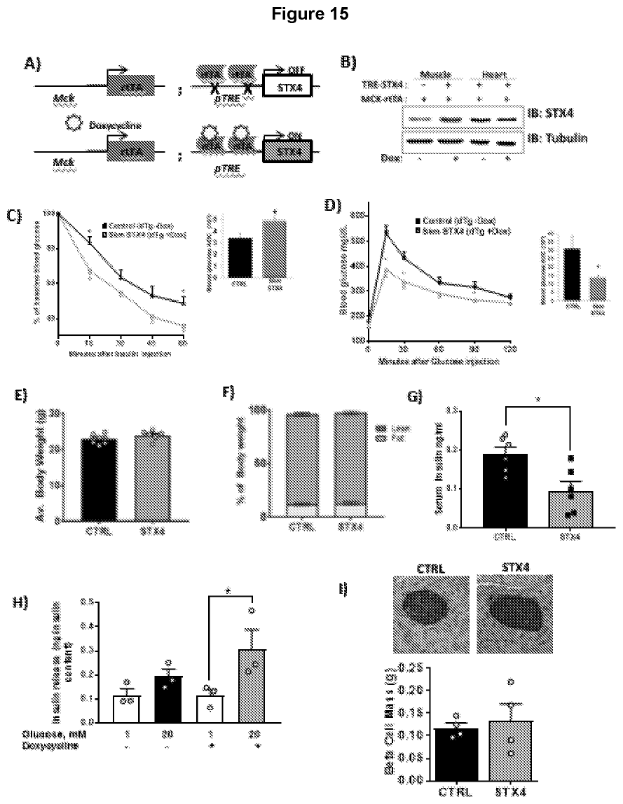

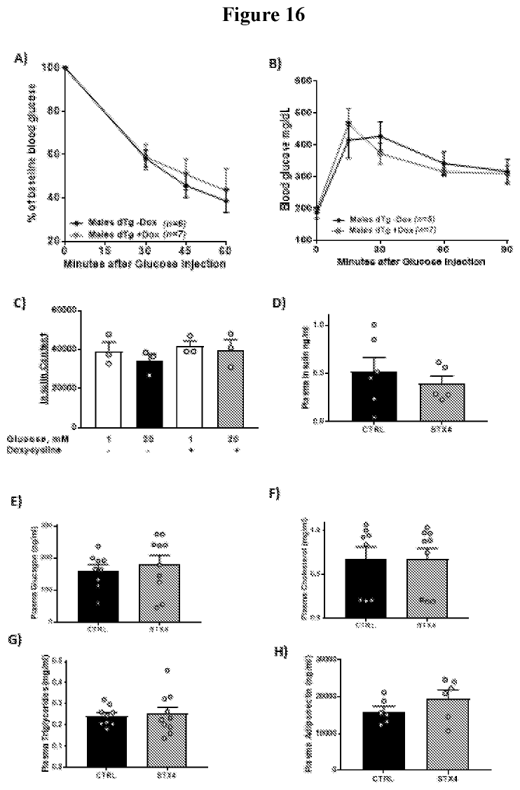

STX4 global overexpressing mice had improved insulin sensitivity and glucose tolerance. In addition, the mice showed increased glucose uptake and GLUT4 translocation in the skeletal muscle (22, 44). However, the mechanistic details of STX4 function in skeletal muscle are insufficient due to lack of tissue specific models. To delineate the skeletal muscle specific mechanisms of STX4, a skeletal muscle specific STX4 enrichment model was generated. A STX4 skeletal muscle specific expression model (SkmSTX4) that selectively drives expression of STX4 in the skeletal muscle upon doxycycline (Dox) treatment in the food or water was generated using a Tet-on inducible system (FIG. 15A). The tissue specificity was achieved by using a modified muscle creatine kinase (Mck) promoter that led to expression in the skeletal muscle but not the heart, as published previously (48). Induction of the transgene with Dox led to a 2-fold increase in STX4 protein expression in the mouse skeletal muscle (FIG. 15B) whereas the heart showed little to no difference in STX4 expression. Once mice were generated, females and males were assessed for insulin sensitivity (intraperitoneal insulin tolerance test, IPITT) and glucose tolerance (intraperitoneal glucose tolerance test, IPGTT). Male double transgenic mice showed no significant change in glucose tolerance and insulin sensitivity (FIGS. 16A-16B), this is unsurprising as male mice are less insulin sensitive than females (49). Female double transgenic mice were either given doxycycline in the water at 2 mg/ml to induce STX4 expression in the muscle (SkmSTX4) or maintained on regular water (CTRL). The SkmSTX4 females showed significantly improved insulin sensitivity (FIG. 15C), while IPGTT showed a significant improvement in glucose tolerance in the SkmSTX4 compared to CTRL (FIG. 15D). These data recapitulate the phenotype seen in the STX4 global overexpression mice (22) suggesting that STX4 enrichment in the skeletal muscle is the primary driver of insulin sensitivity and glucose homeostasis, without effecting body composition or body weight (FIGS. 15E-15F). Basal serum insulin levels in 6-hour fasted SkmSTX4 mice were slightly lower than CTRL mice (FIG. 15G), but not in 18-hour fasted mice (FIG. 16E). Consistent with lack of effect upon islet function, glucose stimulated insulin secretion (GSIS) ex vivo was normal and even enhanced in skmSTX4 mice relative to CTRL mouse islets; no change in islet insulin content nor changes in beta cell mass were detected (FIG. 16C, and FIGS. 15H-15I). Serum analytes from overnight fasted mice, such as glucagon, cholesterol, triglycerides, and adiponectin showed no significant changes between SkmSTX4 and CTRL mice (FIGS. 16D-16H).

### Example 7: High-Fat Diet (HFD)-Induced Insulin Resistance is Reversible by Skeletal Muscle Specific Delivery of STX4 In Vivo

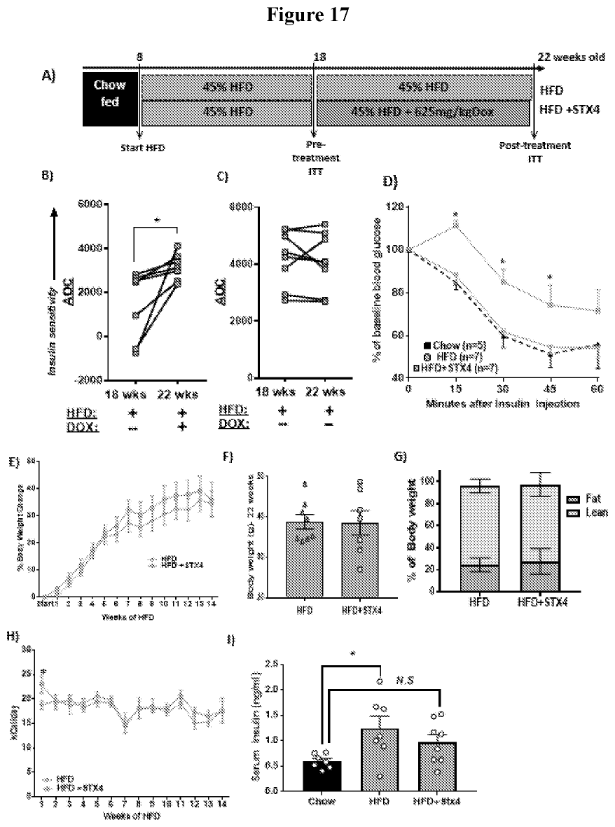

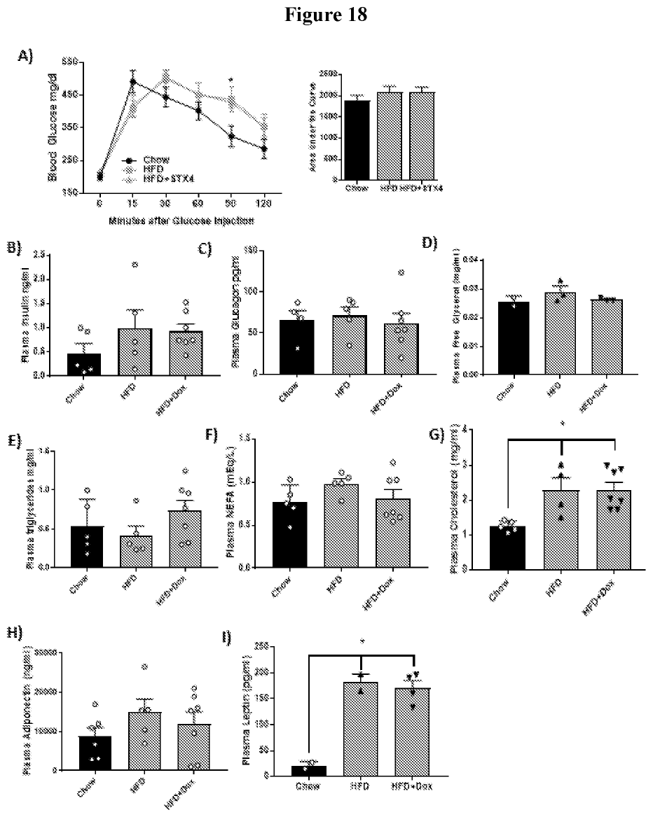

Given that STX4 inducible expression in the skeletal muscle is sufficient to mediate glucose homeostasis, and prevent insulin resistance, whether targeted STX4 enrichment in skeletal muscle tissue is sufficient to remediate insulin resistance induced by high fat diet was investigated. To test this, 8-week old male mice (males were used due to the inability for females to gain sufficient weight on high fat diet feeding to induce insulin resistance (50)) were fed a 45% kcal from fat (high fat diet-HFD) for 10 weeks (FIG. 17A). At 18 weeks of age the mice were evaluated for insulin resistance via IPITT). Insulin resistance was declared in mice that failed to show insulin-lowering of blood glucose levels in 6-hour fasted mice to 60% of initial basal blood glucose levels within 60 minutes. Once insulin resistance was established, the most insulin resistant mice were provided HFD that contained doxycycline (600 mg/kg) to induce STX4 expression in the skeletal muscle (HFD+STX4). CTRL mice were maintained on the high fat diet without STX4 induction (HFD). After 4 weeks of Dox treatment, all the mice were re-assessed for insulin sensitivity (FIG. 17A). A direct comparison of the area over the curve (AOC) for each mouse against itself at 18 weeks and then again at 22 weeks indicated that STX4 induction in the insulin resistant mice at 18 weeks led to increased insulin sensitivity in 100% (7 out of 7) of the mice (FIG. 17B), highlighting the ability of STX4 to remediate insulin resistance in high-fat fed mice, despite their continued intake of a diabetogenic diet. In contrast, 6 out of the 7 CTRL mice that were maintained on HFD without STX4 induction remained or became even more insulin resistant (FIG. 17C). Remarkably, not only do the mice with STX4 induction become more insulin sensitive with STX4 enrichment, but they become as insulin sensitive as age matched chow fed controls (FIG. 17D). This reversal of insulin resistance in the HFD+STX4 mice occurred independent of changes in body weight change over time, terminal body weight, and body fat composition (FIGS. 17E-17G). With that, there is no difference in caloric intake (FIG. 17H). Despite the restoration of peripheral insulin sensitivity, STX4 did not restore glucose tolerance (FIG. 18A), as has been reported previously (Jing et al 2019 and 51). FIG. 17I shows that 6-hour fasted serum insulin levels remain elevated in the HFD+STX4 mice, indicative of HFD-induced beta cell dysfunction, which is known to negatively impact whole body glucose tolerance (52). Similar serum analyte changes driven by HFD were not reversible by STX4 induction, such as cholesterol and Leptin, while others were unchanged by HFD or STX4 induction (FIGS. 18B-18I).

### Example 8: STX4 Induction in HFD Mice Changes Energy Source Utilization, Reverses Sedentary Behavior, and Improves Mitochondrial Function

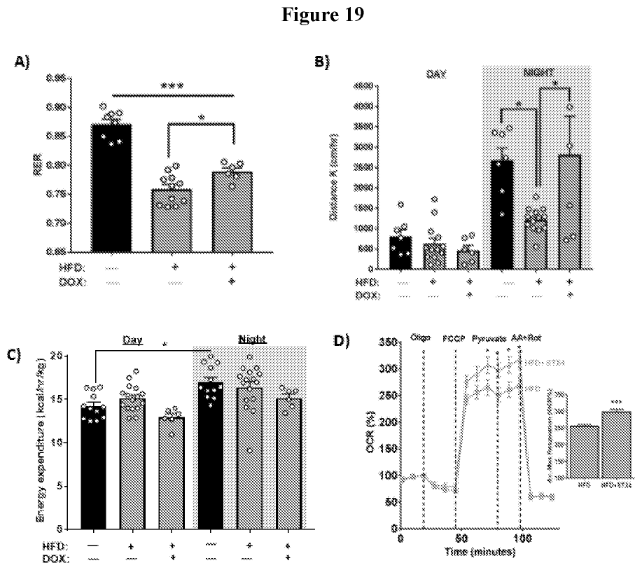

To assess any potential changes in metabolism on animal behaviour with STX4 induction, mice were placed in metabolic caging units to assess their metabolic phenotype. The system was able to capture metabolic parameters of the mice such as food and water intake, the respiratory exchange ratio (RER) as well as voluntary movement (amongst other things). The RER measures the amount of oxygen consumed and carbon dioxide produced by the mice and can indirectly assess the oxidative capacity of mouse muscle (53). The RER can also be used to calculate the respiratory quotient, a parameter indicative of the metabolic fuel source being used to supply energy to the body (54). Chow fed mice had an expected RER of around 0.85 indicated the fuel source is a mixture of carbohydrates and fat, however, upon high fat feeding, the RER decreased to ˜0.7, highlighting the shift to a predominantly fat based source of energy. This change upon high fat feeding was previously reported and was seen as early as 3 days after HFD (55). However, upon STX4 induction there was a significant increase in RER back towards control, reaching a value of ˜0.8 (FIG. 19A), suggesting that STX4 induction caused a metabolic shift in substrate utilization. Spontaneous physical activity (distance K) was characteristically seen to diminish after 10 weeks on HFD compared with the high levels in mice on standard chow diet (FIG. 19B). Remarkably, HFD+STX4 mice displayed robust spontaneous physical activity levels similar to that of chow-fed controls, in contrast to their HFD sedentary counterparts. This change is not due to changes in energy expenditure, other than a significant increase in chow mice between day and night that is lost upon HF-feeding (FIG. 19C). These data suggest that not only is STX4 enrichment conferring a more metabolically healthy phenotype, it is also restoring the spontaneous physical activity levels back to that of young chow fed mice. This is consistent with published data showing that exercise-trained mice exhibit higher levels of spontaneous activity (56).

Given that mitochondrial dysfunction is a well-established consequence of high fat feeding and insulin resistance (57, 58), with high fat diet leading to a decrease in mitochondrial number in human skeletal muscle (59, 60), changes in mitochondrial structure as well as reduction in oxidative phosphorylation and mitochondrial respiration (61-63), if the insulin sensitizing effects of STX4 enrichment in skeletal muscle is due to changes in mitochondria was investigated. Extracellular flux analysis, more commonly known as seahorse analysis (Agilent), was used to assess mitochondrial respiration rate in primary myofibers isolated from the fibrus digitorum brevis (FDB) muscle of high fat fed and with and without STX4 induction (FIG. 19D). Upon isolation and assessment of the myofibers, it was found that STX4 induction in HFD mice, compared to CTRL HFD only mice, led to a significant increase in uncoupled maximal mitochondrial respiration upon injection with FCCP, a protonophore capable of allowing maximal electron transport chain function. This indicates that STX4 is playing a role in modifying mitochondrial function.

### Example 9: STX4 Skeletal Muscle Specific Enrichment Impacts Mitochondrial Morphology

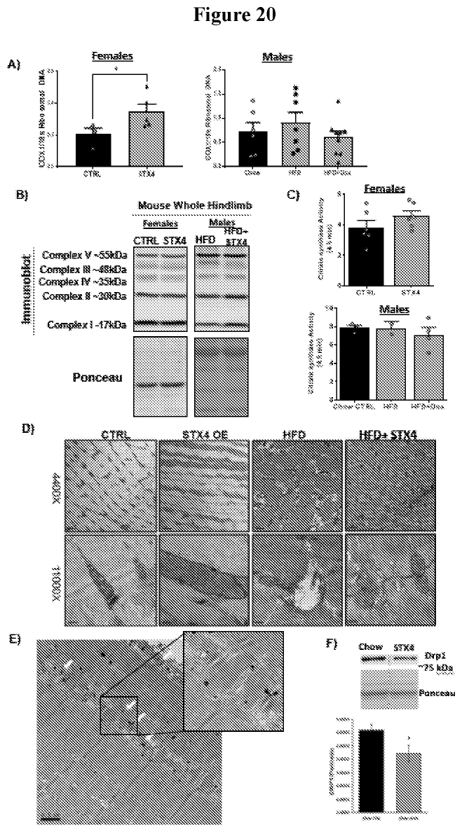

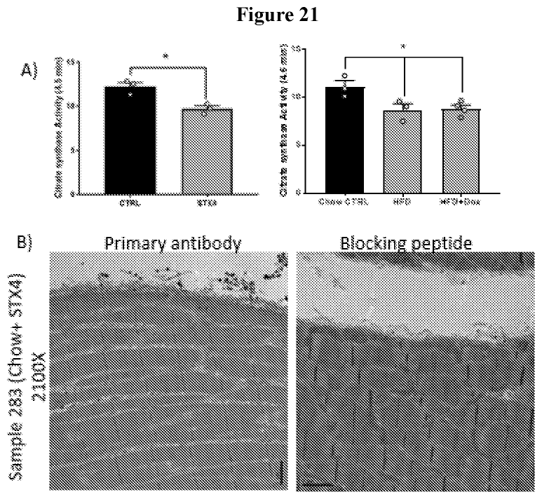

Mitochondrial DNA (mtDNA, detected by COX1 level) to ribosomal DNA ratio in the skeletal muscle showed that while chow-fed female Skm-STX4 muscle elevated mtDNA levels, mtDNA levels amongst HFD+STX4, HFD and chow fed male mice did not differ (FIG. 20A). This suggests that mitochondrial number is not the reason for the increase in mitochondrial respiration in the HF-fed mice. To delineate the exact role of STX4 in mediating mitochondrial respiration, multiple mitochondrial function assays were performed to narrow down the exact mechanism of action. Immunoblotting for the electron transport chain complexes in Chow and HFD muscle tissue lysate indicated no change in protein content of these sub-units (FIG. 20B). Citrate synthase activity, a common enzymatic measure of intact mitochondria, was found to be similar amongst all groups (FIG. 20C). Experiments were conducted in either skeletal muscle hindlimb or tibialis anterior (TA), which had very similar fiber type composition (64). This is important as fiber type alone can have changes on mitochondrial structure and function (65). Furthermore, experiments using mouse TA showed no elevation of CS activity, consistent with no increases seen in mtDNA; if anything, levels of CS activity in chow-fed STX4 induced female mice compared to CTRL TA muscle where slightly reduced, as well as that in HFD and HFD+STX4 mice relative to chow fed male TA muscle (FIG. 21A). Without being bound by any theory, this difference may be related to slight divergence in fiber type between the muscles, and changes in CS activity according to fiber type (66).

Given the lack of functional changes that could account for the improved function in the muscle mitochondria, the structure of the mitochondria in TA muscle was evaluated using transmission electron microscopy (67). Mitochondrial dynamics and structure play a large part in mitochondria function and integrity. Changes in mitochondrial size and structure mediated by changes in fission or fusion, are central to complications in obesity and diabetes, with high fat feeding shown to increase mitochondrial fission and vacuolisation (loss of cristae uniformity, where mitochondria appear to have holes inside them (68)), and exercise, a mediator of prediabetes, shown to increase mitochondrial fusion and elongation (69). Images taken of the chow fed mice induced with STX4 had elongated mitochondria with highly organized and structured cristae. With HF-feeding, the mitochondria appeared fragmented and vacuolised. However, mitochondria in HFD+STX4 muscle appeared very similar to those in chow-fed mice, with much reduced vacuolisation and fragmentation (FIG. 20D). This suggests that STX4 enrichment in healthy conditions increased mitochondrial elongation, and in unhealthy HFD conditions reversed the fragmented phenotype to normal. To understand how STX4, a characteristically plasma membrane localized protein, is eliciting this effect on the mitochondria, the localisation of STX4 in the muscle was evaluated. Immunogold labeling revealed that STX4 was localized not only to the plasma membrane of fibre bundles, but also on the outer mitochondrial membrane (FIG. 20E). This was validated using blocking peptide, which inhibited the localisation to the mitochondrial membrane (FIG. 21B). A recent report shows that STX4 can be localized at the mitochondrial membrane in a variety of other cell types (70). The mitochondrial locale of STX4 led to the assessment whether STX4 is mediating mitochondrial dynamics. While STX4 enriched hindlimb muscle from chow-fed female mice showed similar mRNA levels of mitochondrial factors (Table 6), a significant decrease in Drp1 protein, a mitochondrial fission factor, was observed (FIG. 20G), causing a decrease in the fusion/fission ratio that could account for the elongated phenotype of the chow-fed female Skm-STX4 mitochondria. Elongated mitochondria are also a hallmark of exercise-trained mice (69).

TABLE 6
mRNA analysis of
mitochondrial dynamics genes

| Gene | Fold Change |
|---|---|
| Drp1 | 1.00 |
| MFN1 | 0.99 |
| MFN2 | 0.97 |
| Fis1 | 1.03 |
| TFAM | 0.903 |
| ATG7 | 1.06 |

Data represent the average ±SE of whole hindlimb skeletal muscle mRNA extracted from 6 female STX4 and CTRL mice.

As stated above, the foregoing are merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCIES

1. Donath M Y, and Shoelson S E. Type 2 diabetes as an inflammatory disease. Nat Rev Immunol. 2011;11(2):98-107.
2. Eizirik D L, Colli M L, and Ortis F. The role of inflammation in insulitis and beta-cell loss in type 1 diabetes. Nat Rev Endocrinol. 2009;5(4):219-26.
3. Ferrannini E, Mari A, Nofrate V, Sosenko J M, and Skyler J S. Progression to diabetes in relatives of type 1 diabetic patients: mechanisms and mode of onset. Diabetes. 2010;59(3):679-85.
4. Andersson S A, Olsson A H, Esguerra JL, Heimann E, Ladenvall C, Edlund A, Salehi A, Taneera J, Degerman E, Groop L, et al. Reduced insulin secretion correlates with decreased expression of exocytotic genes in pancreatic islets from patients with type 2 diabetes. Mol Cell Endocrinol. 2012;364(1-2):36-45.
5. Ostenson C G, Gaisano H, Sheu L, Tibell A, and Bartfai T. Impaired gene and protein expression of exocytotic soluble N-ethylmaleimide attachment protein receptor complex proteins in pancreatic islets of type 2 diabetic patients. Diabetes. 2006;55(2):435-40.
6. Nagamatsu S, Nakamichi Y, Yamamura C, Matsushima S, Watanabe T, Ozawa S, Furukawa H, and Ishida H. Decreased expression of t-SNARE, syntaxin 1, and SNAP-25 in pancreatic beta-cells is involved in impaired insulin secretion from diabetic GK rat islets: restoration of decreased t-SNARE proteins improves impaired insulin secretion. Diabetes. 1999;48(12):2367-73.
7. Choi D, Radziszewska A, Schroer S A, Liadis N, Liu Y, Zhang Y, Lam PP, Sheu L, Hao Z, Gaisano H Y, et al. Deletion of Fas in the pancreatic beta-cells leads to enhanced insulin secretion. Am J Physiol Endocrinol Metab. 2009;297(6):E1304-12.
8. Liang T, Qin T, Xie L, Dolai S, Zhu D, Prentice K J, Wheeler M, Kang Y, Osborne L, and Gaisano H Y. New Roles of Syntaxin-1A in Insulin Granule Exocytosis and Replenishment. J Biol Chem. 2017;292(6):2203-16.
9. Oh E, Stull N D, Mirmira R G, and Thurmond D C. Syntaxin 4 up-regulation increases efficiency of insulin release in pancreatic islets from humans with and without type 2 diabetes mellitus. J Clin Endocrinol Metab. 2014;99(5):E866-70.
10. Sadoul K, Lang J, Montecucco C, Weller U, Regazzi R, Catsicas S, Wollheim C B, and Halban P A. SNAP-25 is expressed in islets of Langerhans and is involved in insulin release. J Cell Biol. 1995;128(6):1019-28.
11. Berchtold L A, Storling Z M, Ortis F, Lage K, Bang-Berthelsen C, Bergholdt R, Hald J, Brorsson C A, Eizirik D L, Pociot F, et al. Huntingtin-interacting protein 14 is a type 1 diabetes candidate protein regulating insulin secretion and beta-cell apoptosis. Proc Natl Acad Sci U S A. 2011;108(37): E681-8.
12. Keller M P, Choi Y, Wang P, Davis D B, Rabaglia M E, Oler A T, Stapleton D S, Argmann C, Schueler K L, Edwards S, et al. A gene expression network model of type 2 diabetes links cell cycle regulation in islets with diabetes susceptibility. Genome Res. 2008;18(5): 706-16.
13. Yang C, Coker K J, Kim J K, Mora S, Thurmond D C, Davis A C, Yang B, Williamson R A, Shulman G I, and Pessin J E. Syntaxin 4 heterozygous knockout mice develop muscle insulin resistance. J Clin Invest. 2001;107(10):1311-8.
14. Spurlin B A, and Thurmond D C. Syntaxin 4 Facilitates Biphasic Glucose-Stimulated Insulin Secretion from Pancreatic Beta Cells. Mol Endocrinol. 2006;20(1):183-93.
15. Kusminski C M, Chen S, Ye R, Sun K, Wang Q A, Spurgin S B, Sanders P E, Brozinick J T, Geldenhuys W J, Li W H, et al. MitoNEET-Parkin Effects in Pancreatic alpha- and beta-Cells, Cellular Survival, and Intrainsular Cross Talk. Diabetes. 2016; 65(6):1534-55.
16. Ye R, Wang M, Wang Q A, Spurgin S B, Wang Z V, Sun K, and Scherer P E. Autonomous interconversion between adult pancreatic alpha-cells and beta-cells after differential metabolic challenges. Mol Metab. 2016;5(7):437-48.
17. Thurmond D C, Ceresa B P, Okada S, Elmendorf J S, Coker K, and Pessin J E. Regulation of insulin-stimulated GLUT4 translocation by muncl8c in 3T3L1 adipocytes. J Biol Chem. 1998;273:33876-83.
18. Wiseman D A, Kalwat M A, and Thurmond D C. Stimulus-induced S-nitrosylation of syntaxin 4 impacts insulin granule exocytosis. J Biol Chem. 2011;286(18):16344-54.
19. Ahn M, Yoder S M, Wang Z, Oh E, Ramalingam L, Tunduguru R, and Thurmond D C. The p21-Activated Kinase (PAK1) is Involved in Diet-Induced Beta Cell Mass Expansion and Survival. Diabetologia. 2016;59(10):2145-55.
20. Chen Y C, Colvin E S, Griffin K E, Maier B F, and Fueger P T. Mig6 haploinsufficiency protects mice against streptozotocin-induced diabetes. Diabetologia. 2014;57(10):2066-75.
21. Oh E, and Thurmond D C. Munc18c Depletion Selectively Impairs the Sustained Phase of Insulin Release. Diabetes. 2009;58: 1165-74.
22. Oh E, Miller R A, and Thurmond D C. Syntaxin 4 overexpression ameliorates effects of aging and high fat diet on glucose control, and may increase lifespan Cell Metab. 2015;22: 499-507.
23. Becker T C, Noel R J, Coats W S, Gomez-Foix A M, Alam T, Gerard R D, and Newgard C B. Use of recombinant adenovirus for metabolic engineering of mammalian cells. Methods Cell Biol. 1994;43 Pt A(161-89).
24. Ravassard P, Hazhouz Y, Pechberty S, Bricout-Neveu E, Armanet M, Czernichow P, and Scharfmann R. A genetically engineered human pancreatic beta cell line exhibiting glucose-inducible insulin secretion. J Clin Invest. 2011; 121(9):3589-97.
25. Oh E, Kalwat M A, Kim M J, Verhage M, and Thurmond D C. Munc18-1 regulates first-phase insulin release by promoting granule docking to multiple syntaxin isoforms. J Biol Chem. 2012;287(31):25821-33.
26. Sarkar S A, Lee C E, Victorino F, Nguyen T T, Walters J A, Burrack A, Eberlein J, Hildemann S K, and Homann D. Expression and regulation of chemokines in murine and human type 1 diabetes. Diabetes. 2012;61(2):436-46.
27. Roep B O, Kleijwegt F S, van Halteren A G, Bonato V, Boggi U, Vendrame F, Marchetti P, and Dotta F. Islet inflammation and CXCL10 in recent-onset type 1 diabetes. Clin Exp Immunol. 2010;159(3):338-43.
28. Homann D. Back From the Brink: The Uses of Targeting the CXCL10:CXCR3 Axis in Type 1 Diabetes. Diabetes. 2015;64(12):3990-2.
29. Lasch S, Muller P, Bayer M, Pfeilschifter J M, Luster A D, Hintermann E, and Christen U. Anti-CD3/Anti-CXCL10 Antibody Combination Therapy Induces a Persistent Remission of Type 1 Diabetes in Two Mouse Models. Diabetes. 2015;64(12):4198-211.
30. Burke S J, Goff M R, Lu D, Proud D, Karlstad M D, and Collier J J. Synergistic expression of the CXCL10 gene in response to IL-1beta and IFN-gamma involves NF-kappaB, phosphorylation of STAT1 at Tyr701, and acetylation of histones H3 and H4. J Immunol. 2013;191(1):323-36.
31. Ortis F, Pirot P, Naamane N, Kreins A Y, Rasschaert J, Moore F, Theatre E, Verhaeghe C, Magnusson N E, Chariot A, et al. Induction of nuclear factor-kappaB and its downstream genes by TNF-alpha and IL-1beta has a pro-apoptotic role in pancreatic beta cells. Diabetologia. 2008;51(7):1213-25.
32. Abdelli S, Ansite J, Roduit R, Borsello T, Matsumoto I, Sawada T, Allaman-Pillet N, Henry H, Beckmann J S, Hering B J, et al. Intracellular stress signaling pathways activated during human islet preparation and following acute cytokine exposure. Diabetes. 2004; 53(11):2815-23.
33. Brozzi F, and Eizirik DL. ER stress and the decline and fall of pancreatic beta cells in type 1 diabetes. Ups J Med Sci. 2016;121(2):133-9.
34. Kracht M J, van Lummel M, Nikolic T, Joosten A M, Laban S, van der Slik A R, van Veelen P A, Carlotti F, de Koning E J, Hoeben R C, et al. Autoimmunity against a defective ribosomal insulin gene product in type 1 diabetes. Nat Med. 2017;23(4):501-7.
35. US Department of Health and Human Services, C. 2017. CDC. National diabetes statistics report, 2017. Atlanta, Ga.
36. Bullard, K. M., Cowie, C. C., Lessem, S. E., Saydah, S. H., Menke, A., Geiss, L. S., Orchard, T. J., Rolka, D. B., and Imperatore, G. 2018. Prevalence of Diagnosed Diabetes in Adults by Diabetes Type—United States, 2016. MMWR Morb Mortal Wkly Rep 67:359-361.
37. Mellbin, L. G., Anselmino, M., and Ryden, L. 2010. Diabetes, prediabetes and cardiovascular risk. Eur J Cardiovasc Prey Rehabil 17 Suppl 1:S9-14.
38. 2011. Standards of Medical Care in Diabetes—2011. Diabetes Care 34:S11-S61.
39. Tuso, P. 2014. Prediabetes and Lifestyle Modification: Time to Prevent a Preventable Disease. The Permanente Journal 18:88-93.
40. Giannarelli, R., Aragona, M., Coppelli, A., and Del Prato, S. 2003. Reducing insulin resistance with metformin: the evidence today. Diabetes & Metabolism 29:6S28-26S35.
41. Volchuk, A., Wang, Q., Ewart, H. S., Liu, Z., He, L., Bennett, M. K., and Klip, A. 1996. Syntaxin 4 in 3T3-L1 adipocytes: regulation by insulin and participation in insulin-dependent glucose transport. Mol Biol Cell 7:1075-1082.
42. Vicogne, J., Vollenweider, D., Smith, J. R., Huang, P., Frohman, M. A., and Pessin, J. E. 2006. Asymmetric phospholipid distribution drives in vitro reconstituted SNARE-dependent membrane fusion. Proceedings of the National Academy of Sciences 103:14761-14766.
43. Khan, A. H., Thurmond, D. C., Yang, C., Ceresa, B. P., Sigmund, C. D., and Pessin, J. E. 2001. Munc18c regulates insulin-stimulated glut4 translocation to the transverse tubules in skeletal muscle. J Biol Chem 276:4063-4069.
44. Spurlin, B. A., Park, S. Y., Nevins, A. K., Kim, J. K., and Thurmond, D. C. 2004. Syntaxin 4 transgenic mice exhibit enhanced insulin-mediated glucose uptake in skeletal muscle. Diabetes 53:2223-2231.
45. Hojman, P., Eriksen, J., and Gehl, J. 2007. Tet-On Induction with Doxycycline after Gene Transfer in Mice: Sweetening of Drinking Water is not a Good Idea. Animal Biotechnology 18:183-188.
46. Bergman, B. C., Cornier, M. A., Horton, T. J., Bessesen, D. H., and Eckel, R. H. 2008. Skeletal muscle munc18c and syntaxin 4 in human obesity. Nutr Metab (Lond) 5:21.
47. Jewell, J. L., Oh, E., and Thurmond, D. C. 2010. Exocytosis mechanisms underlying insulin release and glucose uptake: conserved roles for Munc18c and syntaxin 4. Am J Physiol Regul Integr Comp Physiol 298:R517-531.
48. Grill, M. A., Bales, M. A., Fought, A. N., Rosburg, K. C., Munger, S. J., and Antin, P. B. 2003. Tetracycline-inducible system for regulation of skeletal muscle-specific gene expression in transgenic mice. Transgenic Res 12:33-43.
49. Amrani, A., Durant, S., Throsby, M., Coulaud, J., Dardenne, M., and Homo-Delarche, F.o. 1998. Glucose Homeostasis in the Nonobese Diabetic Mouse at the Prediabetic Stage*. Endocrinology 139:1115-1124.
50. Pettersson, U. S., Waldén, T. B., Carlsson, P.-O., Jansson, L., and Phillipson, M. 2012. Female Mice are Protected against High-Fat Diet Induced Metabolic Syndrome and Increase the Regulatory T Cell Population in Adipose Tissue. PLOS ONE 7:e46057.
51. Brachs, S., Polack, J., Brachs, M., Jahn-Hofmann, K., Elvert, R., Pfenninger, A., Bärenz, F., Margerie, D., Mai, K., Spranger, J., et al. 2018. Genetic Nicotinamide N-Methyltransferase (<em>Nnmt</em>) Deficiency in Male Mice Improves Insulin Sensitivity in Diet-Induced Obesity but Does Not Affect Glucose Tolerance. Diabetes:db180780.
52. Bruin, Jennifer E., Saber, N., Braun, N., Fox, Jessica K., Mojibian, M., Asadi, A., Drohan, C., O'Dwyer, S., Rosman-Balzer, Diana S., Swiss, Victoria A., et al. 2015. Treating Diet-Induced Diabetes and Obesity with Human Embryonic Stem Cell-Derived Pancreatic Progenitor Cells and Antidiabetic Drugs. Stem Cell Reports 4:605-620.
53. Ramos-Jiménez, A., Hernández-Torres, R. P., Torres-Durán, P. V., Romero-Gonzalez, J., Mascher, D., Posadas-Romero, C., and Juarez-Oropeza, M. A. 2008. The Respiratory Exchange Ratio is Associated with Fitness Indicators Both in Trained and Untrained Men: A Possible Application for People with Reduced Exercise Tolerance. Clinical medicine. Circulatory, respiratory and pulmonary medicine 2:1-9.
54. McClave, S. A., Lowen, C. C., Kleber, M. J., McConnell, J. W., Jung, L. Y., and Goldsmith, L. J. 2003. Clinical use of the respiratory quotient obtained from indirect calorimetry. JPEN J Parenter Enteral Nutr 27:21-26.
55. Marvyn, P. M., Bradley, R. M., Mardian, E. B., Marks, K. A., and Duncan, R. E. 2016. Data on oxygen consumption rate, respiratory exchange ratio, and movement in C57BL/6J female mice on the third day of consuming a high-fat diet. Data in brief 7:472-475.
56. Chow, L. S., Greenlund, L. J., Asmann, Y. W., Short, K. R., McCrady, S. K., Levine, J. A., and Nair, K. S. 2007. Impact of endurance training on murine spontaneous activity, muscle mitochondrial DNA abundance, gene transcripts, and function. J Appl Physiol (1985) 102:1078-1089.
57. Bonnard, C., Durand, A., Peyrol, S., Chanseaume, E., Chauvin, M.-A., Morio, B., Vidal, H., and Rieusset, J. 2008. Mitochondrial dysfunction results from oxidative stress in the skeletal muscle of diet-induced insulin-resistant mice. The Journal of clinical investigation 118:789-800.
58. Miotto, P. M., LeBlanc, P. J., and Holloway, G. P. 2018. High-Fat Diet Causes Mitochondrial Dysfunction as a Result of Impaired ADP Sensitivity. Diabetes 67:2199-2205.
59. Kim, J.-Y., Hickner, R. C., Cortright, R. L., Dohm, G. L., and Houmard, J. A. 2000. Lipid oxidation is reduced in obese human skeletal muscle. American Journal of Physiology-Endocrinology and Metabolism 279:E1039-E1044.
60. Ritov, V. B., Menshikova, E. V., He, J., Ferrell, R. E., Goodpaster, B. H., and Kelley, D. E. 2005. Deficiency of Subsarcolemmal Mitochondria in Obesity and Type 2 Diabetes. Diabetes 54:8-14.
61. Bach, D., Pich, S., Soriano, F. X., Vega, N., Baumgartner, B., Oriola, J., Daugaard, J. R., Lloberas, J., Camps, M., Zierath, J. R., et al. 2003. Mitofusin-2 determines mitochondrial network architecture and mitochondrial metabolism. A novel regulatory mechanism altered in obesity. J Biol Chem 278:17190-17197.
62. Chen, D., Li, X., Zhang, L., Zhu, M., and Gao, L. 2018. A high-fat diet impairs mitochondrial biogenesis, mitochondrial dynamics, and the respiratory chain complex in rat myocardial tissues. J Cell Biochem 119:9602.
63. Putti, R., Sica, R., Migliaccio, V., and Lionetti, L. 2015. Diet impact on mitochondrial bioenergetics and dynamics. Frontiers in physiology 6:109-109.
64. Augusto, V., Padovani, C., Eduardo, G., and Campos, R. 2004. Skeletal muscle fiber types in C57BL6J mice. 89-94 pp.
65. Mishra, P., Varuzhanyan, G., Pham, A. H., and Chan, D. C. 2015. Mitochondrial Dynamics is a Distinguishing Feature of Skeletal Muscle Fiber Types and Regulates Organellar Compartmentalization. Cell metabolism 22:1033-1044.
66. Delp, M. D., and Duan, C. 1996. Composition and size of type I, IIA, IID/X, and IIB fibers and citrate synthase activity of rat muscle. Journal of Applied Physiology 80:261-270.
67. Bujak, A. L., Crane, J. D., Lally, J. S., Ford, R. J., Kang, S. J., Rebalka, I. A., Green, A. E., Kemp, B. E., Hawke, T. J., Schertzer, J. D., et al. 2015. AMPK activation of muscle autophagy prevents fasting-induced hypoglycemia and myopathy during aging. Cell Metab 21:883-890.
68. Luzzo, K.M., Wang, Q., Purcell, S. H., Chi, M., Jimenez, P. T., Grindler, N., Schedl, T., and Moley, K. H. 2012. High Fat Diet Induced Developmental Defects in the Mouse: Oocyte Meiotic Aneuploidy and Fetal Growth Retardation/Brain Defects. PLOS ONE 7:e49217.
69. Axelrod, C. L., Fealy, C. E., Mulya, A., and Kirwan, J. P. 2018. Exercise training remodels human skeletal muscle mitochondrial fission and fusion machinery towards a pro-elongation phenotype. Acta Physiol (Oxf):e13216.
70. Orre, L. M., Vesterlund, M., Pan, Y., Arslan, T., Zhu, Y., Fernandez Woodbridge, A., Frings, O., Fredlund, E., and Lehtiö, J. 2019. SubCellBarCode: Proteome-wide Mapping of Protein Localization and Relocalization. Molecular Cell 73:166-182.e167.
71. Burcelin, R., Crivelli, V., Dacosta, A., Roy-Tirelli, A., and Thorens, B. 2002. Heterogeneous metabolic adaptation of C57BL/6J mice to high-fat diet. Am J Physiol Endocrinol Metab 282: E834-842.
72. Buettner, R., Schölmerich, J., and Bollheimer, L. C. 2007. High-fat Diets: Modeling the Metabolic Disorders of Human Obesity in Rodents. Obesity 15:798-808.
73. Small, L., Brandon, A. E., Turner, N., and Cooney, G. J. 2018. Modeling insulin resistance in rodents by alterations in diet: what have high-fat and high-calorie diets revealed? American Journal of Physiology-Endocrinology and Metabolism 314: E251-E265.
74. Imai, A., Nashida, T., Yoshie, S., and Shimomura, H. 2003. Intracellular localisation of SNARE proteins in rat parotid acinar cells: SNARE complexes on the apical plasma membrane. Archives of Oral Biology 48:597-604.
75. Low, S. H., Vasanji, A., Nanduri, J., He, M., Sharma, N., Koo, M., Drazba, J., and Weimbs, T. 2006. Syntaxins 3 and 4 Are Concentrated in Separate Clusters on the Plasma Membrane before the Establishment of Cell Polarity. Molecular Biology of the Cell 17:977-989.
76. Mandon, B., Chou, C. L., Nielsen, S., and Knepper, M. A. 1996. Syntaxin-4 is localized to the apical plasma membrane of rat renal collecting duct cells: possible role in aquaporin-2 trafficking. The Journal of Clinical Investigation 98:906-913.
77. Dahlmans, D., Houzelle, A., Schrauwen, P., and Hoeks, J. 2016. Mitochondrial dynamics, quality control and miRNA regulation in skeletal muscle: implications for obesity and related metabolic disease. Clin Sci (Lond) 130:843-852.

1. A method of improving the life expectancy of β-cells in a subject comprising overexpressing Stx4 in the β-cells.

2. A method of protecting β-cells in a subject from inflammatory damage comprising overexpressing Stx4 in the β-cells.

3. A method of preserving functional β-cell mass in a subject comprising overexpressing Stx4 in the β-cells.

4. A method of treating an insulin-related disease or condition in a subject comprising overexpressing Stx4 in the β-cells.

5. A method of reversing insulin resistance in a subject comprising overexpressing Stx4 in skeletal muscle of the subject.

6. The method of any one of claims 1-5, further comprising upregulating HSPA6, and/or downregulating one or more genes selected from the group consisting of CXCL9, CXCL10, and CXCL11.

7. The method of any one of claims 1-6, wherein overexpressing Stx4 in the β-cells comprises inducing overexpression of Stx4 under the control of a human insulin promoter.

8. The method of any one of claims 1-7, wherein the β-cells overexpressing Stx4 are transplanted to the subject.

9. The method of any one of claims 1-8, wherein the subject suffers from an insulin-related disease or condition selected from the group consisting of type 1 diabetes, type 2 diabetes, chronic pancreatitis, pancreatectomy, insulin resistance, prediabetes, and age-related insulin resistance.

10. A pharmaceutical composition comprising β-cells overexpressing Stx4, wherein the β-cells have longer life expectancy, lower susceptibility to inflammatory damage, and/or preserving functional β-cell mass, compared to regular β-cells without overexpressing Stx4.

11. The pharmaceutical composition of claim 10, wherein the β-cells comprising a vector comprising a sequence encoding a human insulin promoter and human Stx4 cDNA.


SEQUENCE LISTING (8 sequences)

SEQ ID NO: 1 - DNA, 22 bp, ARTIFICIAL
  STMN4 forward primer
atcctgaagccaccctcctttg

SEQ ID NO: 2 - DNA, 23 bp, ARTIFICIAL
  STMN4 reverse primer
tccctgttctccttgttggattc

SEQ ID NO: 3 - DNA, 25 bp, ARTIFICIAL
  CECAM5 forward primer
agcctcacttctaaccttctggaac

SEQ ID NO: 4 - DNA, 21 bp, ARTIFICIAL
  CECAM5 reverse primer
gtagccaaaaagatgctgggg

SEQ ID NO: 5 - DNA, 27 bp, artificial
  CXCL9 forward primer
actatccacctacaatccttgaaagac

SEQ ID NO: 6 - DNA, 22 bp, Artificial Sequence
  CXCL9 reverse primer
tcacatctgaatctgggtttag

SEQ ID NO: 7 - DNA, 26 bp, Artificial Sequence
  CXCL10 forward primer
agcaaggaaaggtctaaaagatctcc

SEQ ID NO: 8 - DNA, 25 bp, Artificial Sequence
  CXCL10 reverse primer
ggcttgacatatactccatgtaggg